(19) United States
(12) Patent Application Publication
(10) Pub. No.: US 2026/0218054 A1
(43) Pub. Date: Jul. 30, 2026

(54) EMULSIFIER AND EMULSION STABILIZER COMPRISING CROSSLINKED POLYESTER ELASTOMER

(71) Applicant: Momentive Performance Materials Inc., Niskayuna, NY (US)

(72) Inventors: Xu QIN, Northvale, NJ (US); Sarah Peeler BLACK, New York, NY (US); Sheena XING, Shanghai (CN); Benjamin FALK, Yorktown Heights, NY (US); Linda WEI, Guangzhou (CN)

(21) Appl. No.: 19/427,716

(22) Filed: Dec. 19, 2025

(30) Foreign Application Priority Data
Dec. 20, 2024 (WO) PCT/CN2024/140853

(51) Int. Cl. C09K 23/36; A61K 8/06; A61K 8/36; A61K 8/81; C09K 23/00

(52) U.S. Cl. CPC C09K 23/36; A61K 8/062; A61K 8/064; A61K 8/361; A61K 8/8152; C09K 23/018; A61K 2800/594

(57) ABSTRACT

The present invention provides an emulsifier or emulsion stabilizer for the preparation of emulsions, comprising at least one crosslinked polyester emulsifying elastomer component (A), and optionally at least one solvent or emollient (B), and optionally one or more emulsifiers or emulsion stabilizers different from components (A) or (B). The invention further relates to cosmetic or personal care compositions, comprising one or more of said emulsifiers or emulsion stabilizers, the of said emulsifiers or emulsion stabilizers for the manufacture of a cosmetic or personal care compositions, and the use a crosslinked polyester emulsifying elastomer component (A), and optionally at least one solvent or emollient (B) as an emulsifier or emulsion stabilizer for the preparation of preferably cosmetic of personal care emulsions, such as skin care products, e.g. in the form of lotions, creams and the like.

The present invention provides an emulsifier or emulsion stabilizer for the preparation of emulsions, comprising at least one crosslinked polyester emulsifying elastomer component (A), and optionally at least one solvent or emollient (B), and optionally one or more emulsifiers or emulsion stabilizers different from components (A) or (B). The invention further relates to cosmetic or personal care compositions, comprising one or more of said emulsifiers or emulsion stabilizers, the of said emulsifiers or emulsion stabilizers for the manufacture of a cosmetic or personal care compositions, and the use a crosslinked polyester emulsifying elastomer component (A), and optionally at least one solvent or emollient (B) as an emulsifier or emulsion stabilizer for the preparation of preferably cosmetic of personal care emulsions, such as skin care products, e.g. in the form of lotions, creams and the like.

### STATEMENT OF THE PROBLEM

Oil-in-water and water-in-oil emulsions are widely used in the personal care and cosmetic industry. Emulsifying silicone elastomer makes it possible to obtain a water-in-oil emulsion with good stability. It is also believed to improve the application properties of the emulsion, and provide a very soft and matting feel after application. Traditional silicone elastomers have limited versatility in terms of compatibility with certain ingredients. In addition, silicones have also been implicated as potentially not biodegradable and being harmful to the environment. Therefore, although the performance of silicone elastomers is unparalleled, there is a demand for alternatives to emulsifying silicone elastomers for use in cosmetic formulations and personal care products.

### STATE OF THE ART

The personal care industry thrives on being able to deliver multiple performance products based on mixtures of several components, with each having performance characteristics important to or desirable in the final formulation. Oil-in-water and water-in-oil emulsions are widely used in the personal care and cosmetic industry to deliver ingredients to skin and hair, either by direct application lotions or creams, or through the medium of wet wipes and other similar applicators. Personal care emulsion products such as creams and lotions desirably have a number of properties in combination: stability in manufacture, formulation, storage and use; a viscosity appropriate to the end use; and preferably a desirable body and good skin feel.

Silicone elastomers are particularly important because they can form elastic particles of three-dimensional polymeric dimethicone and provide a beneficial sensory, texture, and optical effect to cosmetic products. As one type of silicone elastomer, the emulsifying silicone elastomer is a highly cross-linked three-dimensional silicone polymer having an elastomeric function coming from the three-dimensional structure, and also an emulsifying function coming from the hydrophobic alkyl group and hydrophilic polyoxyalkylene group or polyglycerol group in the structure. While the definition “emulsifying silicone elastomer” is used herein to describe the dominating characteristic of the component in comparison with other silicone elastomers as later described, this does not mean the component is devoid of other functions such as gelling upon absorbing solvent.

Emulsifying silicone elastomer makes it possible to obtain a water-in-oil emulsion with good stability. It is also believed to improve the application properties of the emulsion, and provide a very soft and matting feel after application. U.S. Pat. No. 9,023,328B2 reported an emulsifying silicone elastomer gel comprising organopolysiloxane polymer having a glycerol derivative and a liquid oil blended with this polymer. A variety of cosmetic compositions comprising at least one emulsifying silicone elastomers was reported (see U.S. Pat. Nos. 8,900,608, 9,023,328, 9,445,985, WO2018228783A1 and US20070173599.

However, traditional emulsifying silicone elastomers have limited versatility in terms of compatibility with polar solvents or emollients such as ester oils, plant-based oils, glycerin, and water. In addition, silicones have also been implicated as potentially being harmful to the environment. Therefore, although the performance of emulsifying silicone elastomers is unparalleled, there is a demand for alternatives in the marketplace for non-silicone based emulsifying elastomer materials.

Polyesters are a class of compounds that contain an ester functional group in their polymer chain. The ester group can be hydrolyzed when treated with certain biological catalysts or certain mixed cultures of microorganisms which renders a large number of polyesters biodegradable. There is a growing interest in recent years to design and develop biobased polyesters from renewable resources as emollients, emulsifiers, film formers, or other functional ingredients for personal care applications (see for example, U.S. Pat. Nos. 8,414,906; 9,334,358; 6,540,987; and 7,820,758).

However, no polyester based emulsifying elastomer or polyester based emulsifying elastomer gel has yet been reported that provides multiple benefits to consumers.

### DETAILED DESCRIPTION OF THE INVENTION

Surprisingly it has been found that a crosslinked polyester component, comprising a crosslinked polyester and optionally one or more solvents or emollients is suitable as emulsifier or emulsion stabilizer for the preparation of emulsions, in particular cosmetic emulsions.

The present invention accordingly provides an emulsifier or emulsion stabilizer for the preparation of emulsions, comprising:
(A) at least one crosslinked polyester component, comprising a crosslinked polyester which is reaction product of:
   (i) at least one compound selected from a polycarboxylic acid, a polycarboxylic acid ester, and combinations thereof,
   (ii) at least one polyol, and
   (iii) optionally one or more monofunctional component selected from
      (a) a monofunctional carboxylic acid; and
      (b) a monofunctional alcohol, and
(B) optionally one or more solvents or emollients.

Emulsifiers and emulsion stabilizer of the invention are useful in cosmetic formulations, helping to mix water and oil-based ingredients to create stable emulsions, and also to improve texture, enhance skin feel, and promote better delivery of active ingredients such as skin or hair care actives.

As will be described in more detail below the crosslinked polyester component (A) (sometimes called polyester elastomer) can be used as dry powder or as a gel which is obtained by swelling the crosslinked polyester e.g. under shear force in the optional one or more solvents or emollients (B) (such as a low molecular weight emollient) to form a uniform polyester gel or paste having a wide viscosity range. The gel is usually a semi-solid colloid formed by suspending the crosslinked polyester in the solvent. The solid particles of the crosslinked polyester in the gel are supposed to form a three-dimensional network that captures and holds the solvent. The crosslinked polyester gel is usually a stable, jelly-like substance that is soft and flexible. These crosslinked polyester components (A) in particular provided as gels in a solvent deliver superior emulsifying and emulsion stabilization benefits to create stable aqueous emulsions and at the same time can also improve texture, enhance skin feel, and promote better delivery of active ingredients such as skin or hair care actives.

The emulsifier or emulsion stabilizer for the preparation of emulsions preferably comprise:
(A) at least one crosslinked polyester component, comprising a crosslinked polyester which is reaction product of:
   (i) at least one compound selected from a polycarboxylic acid, a polycarboxylic acid ester, and combinations thereof,
   (ii) at least one polyol, and
   (iii) optionally one or more monofunctional component selected from
      (a) a monofunctional carboxylic acid; and
      (b) a monofunctional alcohol,
(B) optionally one or more solvents or emollients, different from (A) or (C), and
(C) optionally one or more emulsifiers or emulsion stabilizers, different from components (A) or (B).

In an aspect the emulsifier or emulsion stabilizer for the preparation of emulsions comprises components (A) and (C), in a further embodiment emulsifier or emulsion stabilizer for the preparation of emulsions comprises components (A), (B) and (C).

In an aspect, the crosslinked polyester component (A) is a reaction product of: compound (i) selected from the group consisting of
(1) one or more poly-carboxylic acids of formula (I)

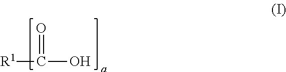

wherein
   R¹ is selected from the group consisting of C2-C52 alkyl group, C2-C52 heteroalkyl group, C2-C52 alkene group, C2-C52 heteroalkene group, C3-C52 cyclic group, or C2-C52 heterocyclic group, and
   a is an integer from 2 to 10, or
(2) one or more carboxylic acid esters of formula (II)

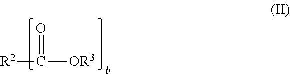

wherein
   R² is selected from the group consisting of C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
   R³ is selected from the group consisting of C1-C22 alkyl group, C2-C22 alkylene group, or C3-C22 cyclic group, and
   b is an integer from 2 to 10, and
(3) a combination (1) and (2) thereof,
compound (ii) selected from one or more polyols of formula (III)

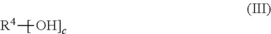

wherein
   R is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   c is an integer from 2 to 10, and
   optional compound (iii) selected from the group consisting of
(a) one or more mono-carboxylic acids of formula (IV)

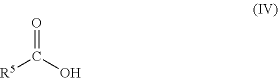

wherein
   R⁵ is C2-C52 alkyl group, C2-C52 heteroalkyl group, C2-C52 alkene group, C2-C52 heteroalkene group, C3-C52 cyclic group, or C2-C52 heterocyclic group,
(b) one or more mono-alcohols of formula (V)

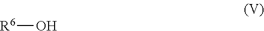

wherein
   R⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the crosslinked polyester component (A) in the emulsifier or emulsion stabilizer for the preparation of emulsions, is reaction product of:
(i) one or more poly-carboxylic acids selected from di-carboxylic acids, tri-carboxylic acids and combinations thereof,
(ii) one or more polyols selected from diols, triols and combinations thereof, and
(iii) optionally one or more monofunctional carboxylic acids.

In an aspect, the crosslinked polyester component (A) in the emulsifier or emulsion stabilizer for the preparation of emulsions, is reaction product of:
(i) one or more dicarboxylic acids,
(ii) one or more triols, and
(iii) optionally one or more monofunctional carboxylic acids.

In an aspect, the crosslinked polyester component (A) in the emulsifier or emulsion stabilizer for the preparation of emulsions, is reaction product of:
(i) one or more poly-carboxylic acids selected from C36 dimer acid, hydrogenated C36 dimer acid, C54 trimer Acid, hydrogenated C54 trimer Acid, citric acid, and combinations thereof,
(ii) one or more polyols selected from glycerol, diglycerol, polyglycerol-3, polyglycerol-4, polyglycerol-6, polyglycerol-10, sugar alcohol, C36 dimer diol, hydrogenated C36 dimer diol, castor oil, hydrogenated castor oil, and combinations thereof, and
(iii) optionally one or more monofunctional carboxylic acids.

In an aspect, the crosslinked polyester component (A) in the emulsifier or emulsion stabilizer for the preparation of emulsions, is reaction product of:
(i) C36 dimer acid, hydrogenated C36 dimer acid, and combinations thereof,
(ii) one or more polyols selected from glycerol, diglycerol, polyglycerol having from 3 to 20 glycerol units, and C36 dimer diol and combinations thereof, and
(iii) optionally one or more monofunctional carboxylic acids.

In an aspect, the preparation of crosslinked polyester component (A) is without solvent or emollient (B). In an aspect, the preparation of crosslinked polyester component (A) is with solvent or emollient (B) as defined herein.

In an aspect, crosslinked polyester component (A) is only comprised of polyester. In an aspect, polyester elastomer is only comprised of crosslinked polyester. In an aspect, crosslinked polyester component (A) is comprised of crosslinked polyester and non-crosslinked polyester.

In an aspect, crosslinked polyester component (A) is comprised of polyester and solvent or emollient (B). In an aspect, crosslinked polyester component (A) is comprised of crosslinked polyester and solvent or emollient (B). In an aspect, the crosslinked polyester component (A) is comprised of crosslinked polyester, non-crosslinked polyester, and solvent or emollient (B).

In an aspect, the crosslinked polyester component (A) is provided as a powder.

The crosslinked polyester component (A) form crosslinked polymer networks. As is well-known to a skilled person in the art such crosslinked polymer are not (completely) soluble and certain methods are available to characterize them, including for example sol-gel analysis determination of the gel fraction), swelling ratio analysis (determination of the swelling ratio), and mechanical analysis (e.g. determination of the modulus) (see e.g. Polym. Chem., 2024, 15, 219-247).

In an aspect, the fraction of the crosslinked polyester component (A) which is not soluble in ethyl acetate (gel fraction) is greater than or equal to 20%. In an aspect, the fraction of polyester elastomer which is not soluble in ethyl acetate (gel fraction) is greater than or equal to 40%. In an aspect, the fraction of the polyester elastomer which is not soluble in ethyl acetate (gel fraction) is greater than or equal to 50%. In an aspect, the fraction of the polyester elastomer which is not soluble in ethyl acetate (gel fraction) is greater than or equal to 60%. In an aspect, the fraction of the polyester elastomer which is not soluble in ethyl acetate (gel fraction) is greater than or equal to 70%. The gel fraction is suitably defined as

$$Gel fraction ( \% ) = 100 \times \frac{weight of dried gel ( insoluble residue of the extraction )}{total weight of the polyester elastomer used in the extraction}$$

In an aspect, the gel fraction can be determined with an extraction method such as the Soxhlet extraction described herein.

In an aspect, crosslinked polyester component (A) is comprised of polyester elastomer and solvent or emollient (B) as defined herein. In an aspect, polyester elastomer composition is comprised of polyester elastomer (A) without solvent or emollient (B) as defined herein. In an aspect, crosslinked polyester component (A) is a gel or a powder.

In an aspect, the crosslinked polyester component (A) is a polyester elastomer combined with one or more solvents or emollients (B), which can be converted to a polyester elastomer gel (the swollen polyester elastomer), for example, by applying a shear force to the composition.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or” where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 6th Ed., Smith, M. B. and March, J., eds. John Wiley & Sons, New York: 2007, the entire contents of which are hereby incorporated by reference.

The term “hydrocarbon”, as used herein by itself or as part of a group, refers to a straight- or branched-chain aliphatic series of one to two hundred carbon atoms, i.e., a C1-C200 hydrocarbon, or the number of carbon atoms designated, e.g., a C1 hydrocarbon such as a methyl, a C2 hydrocarbon such as ethyl, etc. In one embodiment, the hydrocarbon is a C2-C200 hydrocarbon group. In an embodiment, the hydrocarbon is a C6-C60 hydrocarbon group. In an embodiment, the hydrocarbon is a C6-C60 hydrocarbon group. In an embodiment, the hydrocarbon is a C2-C60 hydrocarbon group. In an embodiment, the hydrocarbon is a C5-C22 hydrocarbon group. Examples of hydrocarbon groups include butyl, octyl, decyl, lauryl, cetyl (palmityl), and stearyl.

The term “alkyl”, as used herein by itself or as part of a group, refers to a straight or branched-chain aliphatic hydrocarbon containing one to two hundred carbon atoms, i.e., a C2-C200 alkyl, or the number of carbon atoms designated, e.g., a C1 alkyl such as methyl, a C2 alkyl such as ethyl, etc. In one embodiment, the alkyl is a C2-C200 alkyl group. In another embodiment, the alkyl is a C6-C60 alkyl group. In another embodiment, the alkyl is a C2-C60 alkyl group. In another embodiment, the alkyl is a C5-C22 alkyl group. Examples of alkyl group include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C2-C200 alkylene group. In another embodiment, the alkene group is a C6-C60 alkene group. In another embodiment, the alkene group is a C6-C60 alkene group. In another embodiment, the alkene group is a C2-C60 alkene group. In another embodiment, the alkene group is a C5-C22 alkene group.

The term “alkyne”, as used herein by itself or as part of a group, refers to an alkyl group containing one, two, three, or more carbon-to-carbon triple bonds. In another embodiment, the alkyne is a C2-C200 alkyne group.

The term “cyclic”, as used herein by itself or as part of a group, refers to a stable cyclic compound containing three or more atoms. In an embodiment, the cyclic is a C3-C200 cyclic group. In an embodiment, the cyclic is a C6-C60 cyclic group. In an embodiment, the cyclic is a C5-C22 cyclic group. Examples of cyclic compound include benzene, cyclopentane, and cyclohexane.

The term “heteroalkyl”, as used herein by itself or as part of a group, refers to a stable straight or branched chain alkyl radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C6-C60 heteroalkyl group. In an embodiment, the heteroalkyl is a C2-C60 heteroalkyl group. Examples of heteroalkyl compound include succinyl, adipoyl, and sebacoyl.

The term “heteroalkene”, as used herein by itself or as part of a group, refers to a stable straight or branched chain alkene radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkene is a C6-C60 heteroalkene group. In an embodiment, the heteroalkene is a C2-C60 heteroalkene group. Examples of heteroalkene compound include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. The term “heterocyclic”, as used herein by itself or as part of a group, refers to a stable cyclic compound containing three or more atoms and at least one heteroatom, which can be the same or different, selected from O, N, or S. In an embodiment, the heterocyclic is a C2-C200 heterocyclic group. In an embodiment, the heterocyclic is C6-C60 heterocyclic group.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C2-C200 heterocyclic group. In an embodiment, the heterocyclic is a C6-C60 heterocyclic group. In an embodiment, the heterocyclic is a C5-C22 heterocyclic group. Examples of heterocyclic compounds include furan, oxolane, and thiophene.

As used herein, the term “olefin” refers to any species having at least one ethylenic double bond such as normal and branched chain aliphatic olefins, cycloaliphatic olefins, aryl substituted olefins, and the like. An olefin can comprise terminal double bond(s) (“terminal olefin”) and/or internal double bond(s) (“internal olefin”) and can be cyclic or acyclic, linear or branched, optionally substituted. The total number of carbon atoms can be from 1 to 100, or from 1 to 40; the double bonds can be unsubstituted or mono-, bi-, tri- or tetrasubstituted.

As used herein, the term “polyolefin” refers to a homopolymer or copolymer of ethylene, propylene, butenes and other unsaturated aliphatic hydrocarbons, vinyl esters (e.g. vinyl acetate), or (meth)acrylics (e.g. butyl acrylate, acrylic acid). Generally, the polyolefin will be a polymer of ethylene, propylene or copolymer thereof, or a copolymer of ethylene or propylene with one or more C4-C12 α-olefin aliphatic comonomers.

A gel is a disperse system comprising at least two components: a solid component and a liquid component. The solid component forms a sponge-like, three-dimensional network whose pores are filled by a liquid. The liquid component is thus immobilized in the solid. In the gel of the invention the solid component is a three-dimensional network formed of the cross-linked polyester elastomer, and the liquid component is formed of one or more solvents or emollients as defined herein. A gel is a semi-solid that can have properties ranging from soft and weak to hard and tough. Gels are also defined as a substantially dilute cross-linked system.

An elastomer gel is thus made from the elastomer powders or particles swelled or dispersed in a liquid, such as a solvent or an emollient, to form a gel. The ability to swell is commonly expressed by the swelling ratio as explained herein.

The term “emulsifier” is intended to include an amphiphilic surface-active agent or surfactant promoting the formation and stabilization of an emulsion.

The term “emulsion stabilizer” is defined as a substance preventing the separation of immiscible liquids by creating a stable emulsion and thus suitably prevents or reduces destabilization of an emulsion as explained below. The “stabilization of an emulsion” intends to include that the addition of an “emulsion stabilizer” to an emulsion leads to an improvement of the stability of an emulsion relative to an emulsion which does not contain the “emulsion stabilizer”.

The term “emulsion” is intended to include as a mixture of two or more liquids that are normally immiscible (unmixable or unblendable) owing to liquid-liquid phase separation. Emulsions are part of a more general class of two-phase systems of matter called colloids. Most frequently the emulsions contain a water phase and an oil phase. These emulsions may be either oil-in-water or water-in-oil. Technically, when generating oil-in-water vs. water-in-oil emulsions, one phase (known as the dispersed phase) is mixed into the other (the continuous phase). In other words, one liquid serves as a sort of base into which another liquid is added. When an emulsion is “oil-in-water,” oil is the dispersed phase that is distributed into the continuous phase, water. In a water-in-oil emulsion, water is the dispersed phase that is distributed into the continuous phase, oil. For the purposes of the present invention, “water-in-oil emulsion”, also called inverse emulsion, is intended to indicate any composition consisting of a continuous oily phase in which the aqueous phase is dispersed in the form of droplets so that a macroscopically homogeneous mixture is observed to the naked eye.

The term “emulsifying elastomer” sometimes used intends to include a crosslinked elastomer as amphiphilic surface-active compound, i.e. one having two parts of different polarity which works as an emulsifier or emulsion stabilizer.

The term “destabilization” refers to the processes by which an emulsion loses its uniform, dispersed structure over time, leading to droplet aggregation or phase separation due to insufficient interfacial stabilization between the immiscible phases. Common destabilization phenomena include but are not limited to the following. A) Sedimentation: downward settling of dispersed droplets due to density differences. B) Creaming: upward migration of dispersed droplets due to density differences between phases. C) Flocculation: reversible aggregation of droplets without loss of individual droplet integrity. D) Coalescence: two or more droplets merge irreversibly into larger droplets, resulting in a decrease in total interfacial area and progression toward phase separation.

### A. Components of the Crosslinked Polyester Component (A)

### 1. Poly-Carboxylic Acids (i)

In some aspects, the at least one poly-carboxylic acid may be a compound of formula (VI)

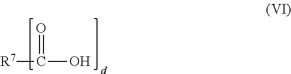

wherein
R⁷ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
d is an integer from 2 to 10.

In a further aspect, the compound is formula (I), wherein R⁷ is C6-C60 alkyl group, C6-C60 heteroalkyl group, C6-C60 alkene group, C6-C60 heteroalkene group, C6-C60 cyclic group, or C6-C60 heterocyclic group; and a is an integer from 2 to 10. In an aspect, the compound is formula (I), wherein a is 2, 3, 4, 5, 6, 7, 8, 9, or 10. In an aspect, the compound is formula (I), wherein a is an integer from 2 to 6. In another aspect, the compound is formula (I), wherein a is 2, 3, 4, 5, or 6.

In an aspect, the polycarboxylic acid is a product of a compound of formula (I), and/or a compound of formula (III) with a compound of formula (V).

In an aspect, the at least one polycarboxylic acid may be selected from the group consisting of citric acid, isocitric acid, aconitic acid, propane-1,2,3-tricarboxylic acid, trimesic acid, carballylic acid, C54 trimer acid, mellitic acid, and combinations thereof. In a further aspect, the at least one polycarboxylic acid may be selected from the group consisting of citric acid, C54 trimer acid, and combinations thereof.

In an aspect, C36 dimer acid is a dicarboxylic acid prepared by dimerizing unsaturated linoleic fatty acid from plant oil.

In an aspect, C54 trimer acid is a polycarboxylic acid prepared by trimerizing unsaturated fatty acids from plant oil.

In some aspects, the unsaturated fatty acids are palmitoleic acid, oleic acid, elaidic acid, vaccenic acid, linoleic acid, linoelaidic acid, or linolenic acid.

In some aspects, the plant oils are soybean oil, safflower oil, linseed oil, corn oil, sunflower oil, olive oil, canola oil, sesame oil, cottonseed oil, palm oil, rapeseed oil, tung oil, peanut oil, or milkweed oil.

In an aspect, the at least one carboxylic acid may be a dicarboxylic acid. In an aspect, the dicarboxylic acid is a compound of formula (VII)

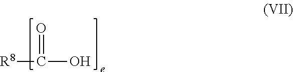

wherein
R⁸ is C2-C200 alkyl group, C2-C200 heteroalkyl group, C2-C200 alkylene group, C2-C200 heteroalkylene group, C2-C200 alkyne group, C2-C200 heteroalkyne group, C3-C200 cyclic group, or C2-C200 heterocyclic group; and
e is 2.

In another aspect, the compound is formula (VII), wherein R⁸ is C2-C200 alkyl group, C2-C200 heteroalkyl group, C2-C200 alkene group, or C2-C200 heteroalkene group and e is 2. In a further aspect, the compound is of formula (VII), wherein R⁸ is C2-C60 alkyl group, C2-C60 heteroalkyl group, C2-C60 alkene group, or C2-C60 heteroalkene group and e is 2. In an aspect, the compound is of formula (VII), wherein R⁸ may be succinyl, adipoyl, sebacoyl, dilinoleyl, or trilinoleyl.

In an aspect, the compound is formula (VII), wherein e may be 1 or 2.

In some aspects, the dicarboxylic acid may be selected from the group consisting of malonic acid, succinic acid, fumaric acid, glutaric acid, adipic acid, pimelic acid, suberic acid, azelaic acid, sebacic acid, undecanedioic acid, dodecanedioic acid, tridecanedioic acid, hexadecanedioic acid, C21 dimer acid, C36 dimer acid, hydrogenated C36 dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C21 dimer acid, C36 dimer acid, hydrogenated C36 dimer acid, and combinations thereof. In one aspect, the dicarboxylic acid is selected from the group consisting of succinic acid, adipic acid, azelaic acid, sebacic acid, undecanedioic acid, dodecanedioic acid, C21 dimer acid, C36 dimer acid, hydrogenated C36 dimer acid, and combinations thereof.

In an aspect, the poly-carboxylic acid is biobased or naturally derived.

### 2. Polycarboxylic Acid Ester (i)

In an aspect, the at least one polycarboxylic acid ester may be a compound of formula (VIII)

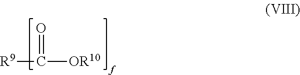

wherein
R⁹ is C1-C22 alkyl group, C2-C22 alkylene group, or C3-C22 cyclic group;
R¹⁰ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
f is an integer from 2 to 10.

In another aspect, the compound is formula (VIII), wherein R⁹ is C1-C22 alkyl group or C2-C22 alkene group; R¹⁰ is C2-C200 alkyl group, C2-C200 heteroalkyl group, C2-C200 alkene group, or C2-C200 heteroalkene group; and f is an integer from 3 to 10.

In a further aspect, the compound is formula (VIII), wherein R⁹ is C1-C10 alkyl group; R¹⁰ is C2-C60 alkyl group, C2-C60 heteroalkyl group, C2-C60 alkene group, or C2-C60 heteroalkene group; and f is an integer from 3 to 10.

In an aspect, the compound is formula (VIII), wherein f is an integer from 3 to 6. In an aspect, the compound is formula (VIII), wherein f is 3, 4, 5, or 6.

In an aspect, the polycarboxylic acid ester is a product of a compound of formula (I), and/or a compound of formula (III) with a compound of formula (V).

In some aspects, the polycarboxylic acid ester may be selected from the group consisting of triethyl citrate, triethyl isocitrate, aconitic acid triethyl ester, propane-1,2,3-tricarboxylic acid triethyl ester, trimesic acid triethyl ester, carballylic acid triethyl ester, C54 trimer acid triethyl ester, mellitic acid hexaethyl ester, and combinations thereof. In a further aspect, the polycarboxylic acid ester may be selected from the group consisting of triethyl citrate, C54 trimer acid triethyl ester, and combinations thereof.

In an aspect, the at least one polycarboxylic acid ester may be a dicarboxylic acid ester. In some aspects, the dicarboxylic acid ester may be a compound of formula (IX)

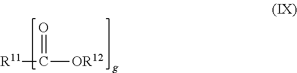

wherein
R¹¹ is C1-C22 alkyl group, C2-C22 alkene group, or C3-C22 cyclic group;
R¹² is C2-C200 alkyl group, C2-C200 heteroalkyl group, C2-C200 alkylene group, C2-C200 heteroalkene group, C2-C200 alkyne group, C2-C200 heteroalkyne group, C3-C200 cyclic group, or C2-C200 heterocyclic group; and
g is 2.

In an aspect, the compound is formula (IX), wherein R¹¹ is C1-C22 alkyl group or C2-C22 alkylene group; R¹² is C2-C200 alkyl group, C2-C200 heteroalkyl group, C2-C200 alkene group, or C2-C200 heteroalkene group; and g is 2. In a further aspect, the compound is formula (IX), wherein R¹¹ is C1-C10 alkyl group; R¹² is C2-C60 alkyl group, C2-C60 heteroalkyl group, C2-C60 alkene group, or C2-C60 heteroalkene group; and g is 2.

In some aspects, the dicarboxylic acid may be selected from the group consisting of diethyl malonate, diethyl succinate, diethyl fumarate, diethyl adipate, diethyl pimelate, diethyl suberate, diethyl azelate, diethyl sebacate, diethyl undecanedioate, diethyl dodecanedioate, diethyl tridecanedioate, diethyl hexadecanedioate, C21 dimer acid diethyl ester, C36 dimer acid diethyl ester, hydrogenated C36 dimer acid diethyl ester, diethyl aspartate, diethyl glutamate, diethyl tartrate, diethyl malate, and combinations thereof. In a further aspect, the dicarboxylic acid ester may be selected from the group consisting of diethyl malonate, diethyl succinate, diethyl adipate, diethyl pimelate, diethyl azelate, diethyl sebacate, diethyl undecanedioate, C21 dimer acid diethyl ester, C36 dimer acid diethyl ester, hydrogenated C36 dimer acid diethyl ester, and combinations thereof. In an aspect, the poly-carboxylic acid ester is biobased or naturally derived.

### 3. Polyol (ii)

In an aspect, the at least one polyol is a compound of formula (X)

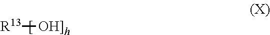

wherein
R¹³ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
h is an integer from 2 to 10.

In an aspect, the compound is formula (X), wherein R¹³ is C2-C200 alkyl group, C2-C200 heteroalkyl group, C2-C200 alkene group, or C2-C200 heteroalkene group; and h is an integer from 2 to 10. In a further aspect, the compound is formula (X), wherein R¹³ is C2-C60 alkyl group, C2-C60 heteroalkyl group, C2-C60 alkene group, or C2-C60 heteroalkene group; and h is an integer from 2 to 10.

In an aspect, the compound is formula (X), wherein h is an integer from 2 to 6. In an aspect, the compound is formula (X), wherein h is 2, 3, 4, 5, or 6.

In an aspect, the polyol is a product of a compound of formula (I), and/or a compound of formula (III) with a compound of formula (V).

In an aspect, the C36 dimer diol is the diol produced from a C36 dimer acid.

In some aspects, the polyol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the polyol may be selected from the group consisting of glycerol, diglycerol, polyglycerol, castor oil, hydrogenated castor oil, sorbitol, gallic acid, and combinations thereof. In another aspect, the alcohol may be selected from the group consisting of glycerol, diglycerol, polyglycerol, castor oil, hydrogenated castor oil, sorbitol, and combinations thereof.

In some aspects, the at least one polyol is a diol. In some aspects, the diol is a compound of formula (XI

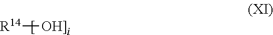

wherein
R¹⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
i is 2.

In an aspect, the compound is formula (XI), wherein R¹⁴ is C2-C200 alkyl group, C2-C200 heteroalkyl group, C2-C200 alkene group, or C2-C200 heteroalkene group; and i is 2. In a further aspect, the compound is formula (XI), wherein R⁴ is C2-C60 alkyl group, C2-C60 heteroalkyl group, C2-C60 alkene group, or C2-C60 heteroalkene group; and i is 2.

In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C36 dimer diol, and combinations thereof. In a further aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof. In one aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof.

In an aspect, the polyol is biobased or naturally derived.

### 4. Mono-Carboxylic Acids

In an aspect, the at least one mono-carboxylic acid is a compound of formula (XII)

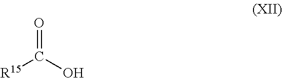

wherein
R¹⁵ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-carboxylic acid is a compound of formula (XII), wherein R¹⁵ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-carboxylic acid is selected from the group consisting of caprylic acid, capric acid, lauric acid, myristic acid, palmitic acid, stearic acid, isostearic acid, behenic acid, oleic acid, linoleic acid, linolenic acid, and combinations thereof. In an aspect, the mono-carboxylic acid is oleic acid. In an aspect, the mono-carboxylic acid is isostearic acid.

In an aspect, R¹⁵ is C2-C52 alkyl group with at least one hydroxyl (—OH) group.

In an aspect, the mono-carboxylic acid is selected from the group consisting of hydroxylstearic acid, ricinoleic acid, isoricinoleic acid, lesquerolic acid, densipolic acid, auricolic acid, dimorphecolic acid, and combinations thereof. In an aspect, the mono-carboxylic acid is hydroxylstearic acid, ricinoleic acid, and combinations thereof.

In an aspect, the mono-carboxylic acid is biobased or naturally derived.

### 5. Mono-Alcohols

In an aspect, the at least one mono-alcohol is a compound of formula (XIII)

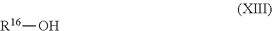

wherein
R¹⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-alcohol is a compound of formula (XIII), wherein R¹⁶ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-alcohol is selected from the group consisting of octanol, decanol, lauryl alcohol, myristyl alcohol, cetyl alcohol, stearyl alcohol, isostearyl alcohol, behenyl alcohol, oleyl alcohol, linoleyl alcohol, linolenyl alcohol, and combinations thereof. In an aspect, the mono-alcohol is oleyl alcohol. In an aspect, the mono-alcohol is isostearyl alcohol.

In an aspect, the mono-alcohol is biobased or naturally derived.

### B. Ratio of Components for the Preparation of Crosslinked Polyester Component (A)

In an aspect, the elastomer comprises a defined molar ratio of carboxyl functional groups (—COOH) from mono-carboxylic acid to hydroxyl functional groups (—OH) from polyol, and from hydroxyl functional groups (—OH) from mono-alcohol to carboxyl functional groups (—COOH) from poly-carboxylic acid. It has been found that the molar ratio of carboxyl functional groups (—COOH) from mono-carboxylic acid to hydroxyl functional groups (—OH) from the polyol and the molar ratio of hydroxyl functional groups (—OH) from mono-alcohol to carboxyl functional groups (—COOH) from poly-carboxylic acid influences the performance of the polyester elastomer and the performance of the gel made from the polyester elastomer.

In an aspect, the molar ratio of carboxyl functional groups (—COOH) from monocarboxylic acid to hydroxyl functional groups (—OH) from polyol is from about 1:2 to about 1:16. In an aspect, the molar ratio of carboxyl functional groups (—COOH) from mono-carboxylic acid to hydroxyl functional groups (—OH) from polyol is from about 1:2 to about 1:14. In an aspect, the molar ratio of carboxyl functional groups (—COOH) from mono-carboxylic acid to hydroxyl functional groups (—OH) from polyol is from about 1:2 to about 1:10. In an aspect, the molar ratio of carboxyl functional groups (—COOH) from mono-carboxylic acid to hydroxyl functional groups (—OH) from polyol is from about 1:2 to about 1:8. In an aspect, the molar ratio of carboxyl functional groups (—COOH) from mono-carboxylic acid to hydroxyl functional groups (—OH) from polyol is from about 1:2 to about 1:5. In an aspect, the molar ratio of carboxyl functional groups (—COOH) from mono-carboxylic acid to hydroxyl functional groups (—OH) from polyol is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of hydroxyl functional groups (—OH) from mono-alcohol to carboxyl functional groups (—COOH) from poly-carboxylic acid is from about 1:2 to about 1:16. In an aspect, the molar ratio of hydroxyl functional groups (—OH) from mono-alcohol to carboxyl functional groups (—COOH) from poly-carboxylic acid is from about 1:2 to about 1:14. In an aspect, the molar ratio of hydroxyl functional groups (—OH) from mono-alcohol to carboxyl functional groups (—COOH) from poly-carboxylic acid is from about 1:2 to about 1:10. In an aspect, the molar ratio of hydroxyl functional groups (—OH) from mono-alcohol to carboxyl functional groups (—COOH) from poly-carboxylic acid is from about 1:2 to about 1:8. In an aspect, the molar ratio of hydroxyl functional groups (—OH) from mono-alcohol to carboxyl functional groups (—COOH) from poly-carboxylic acid is from about 1:2 to about 1:5. In an aspect, the molar ratio of hydroxyl functional groups (—OH) from mono-alcohol to carboxyl functional groups (—COOH) from poly-carboxylic acid is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid to hydroxyl functional groups (—OH) from polyol is from about 2:1 to about 1:4. In an aspect, the ratio of carboxyl functional groups (—COOH) from poly-carboxylic acid to hydroxyl functional groups (—OH) from polyol is from about 2:1 to about 1:2. In an aspect, the ratio of carboxyl functional groups (—COOH) from poly-carboxylic acid to hydroxyl functional groups (—OH) from polyol is from about 1.5:1 to about 1:1.5. In an aspect, the ratio of carboxyl functional groups (—COOH) from poly-carboxylic acid to hydroxyl functional groups (—OH) from polyol is from about 1.5:1 to about 1:1.25. In an aspect, the ratio of carboxyl functional groups (—COOH) from poly-carboxylic acid to hydroxyl functional groups (—OH) from polyol is about 1:2, about 2:3, about 1:1, about 2:1, about 1:4, or about 1:3.

In an aspect, the conversion of carboxylic acid functional groups (—COOH) to ester functional groups (—CO(O)—) is no less than 80% by mole. The percent conversion is calculated by titration of carboxylic acid functional groups (—COOH) with 0.1 N KOH in isopropanol.

### C. Methods of Preparing the Polyester Elastomer (A)

### 1. Esterification Reaction

In one aspect, the present disclosure is directed to a method of preparing a polyester elastomer (A) comprising reacting:
(i) at least one poly-carboxylic acid, at least one poly-carboxylic acid ester, or a combination thereof, and
(ii) at least one polyol; and
(iii)(a) optional one or more mono-carboxylic acids; and
(iii)(b) optional one or more mono-alcohols.

In an aspect, the elastomer prepared is a polyester elastomer (A). In an aspect, the elastomer prepared is a crosslinked polyester elastomer.

In an aspect, the preparation of the polyester elastomer (A) is under nitrogen protection, is under vacuum, or is a combination thereof.

In an aspect, a polyester elastomer is prepared by reacting:
(i) at least one poly-carboxylic acid, at least one poly-carboxylic acid ester, or a combination thereof, and
(ii) at least one polyol; and
(iii)(a) one or more mono-carboxylic acids; or
(iii)(b) one or more mono-alcohols.

In an aspect, a polyester elastomer is prepared by reacting:
(i) at least one poly-carboxylic acid, at least one poly-carboxylic acid ester, or a combination thereof, and
(ii) at least one polyol; and
(iii)(a) one or more mono-carboxylic acids.

In an aspect, the crosslinked polyester component (A) is reaction product of:
(i) one or more poly-carboxylic acids selected from di-carboxylic acids, tri-carboxylic acids and combinations thereof,
(ii) one or more polyols selected from diols, triols and combinations thereof, and
(iii) (a) optionally one or more monofunctional carboxylic acids.

In an aspect, the crosslinked polyester (A) is reaction product of:
(i) one or more dicarboxylic acids
(ii) one or more triols, and
(iii) (a) optionally one or more monofunctional carboxylic acids.

Preferably a mono-carboxylic acid is used in the preparation of the polyester elastomer (A).

In an aspect, wherein the reaction comprised an activated di-carboxylic acid or tricarboxylic acid, the preparation of the elastomer further comprises addition of water to quench the activating agent from the reaction.

In an aspect, esterification is conducted in solvent or emollient. In another aspect, esterification is conducted in more than one solvent or emollient (B).

In an aspect, esterification is carried out in the absence of a solvent or emollient (B).

In an aspect the crosslinked polyester component (A) is obtained by a process comprising the steps of:
(a) reacting compounds (i), (ii) and optionally (iii) as defined above,
   optionally in the presence of one or more solvents or emollients (B),
   to obtain a cross-linked polyester or a mixture thereof with the optional components,
(b) optionally subjecting the crosslinked polyester or a mixture thereof with the optional component (B) obtained in step (a) to a mechanical comminution process to obtain a cross-linked polyester component (A), or
(c) optionally adding one or more (further) solvents (B) to the crosslinked polyester (or a mixture thereof with the optional component (B)) obtained in step (a), allowing the resulting mixture to swell in said solvent, subjecting said swollen mixture to a mechanical comminution process and/or homogenization process in a reactor with planetary mixers to obtain a cross-linked polyester component (A) as a gel. In an aspect step (b) is carried out by mechanical stirring. In an aspect the mechanical comminution process step (c) is carried out with a three-roll mill.

The shearing process in steps (b) or (c) above is suitably carried out with a batch mixer, planetary mixer, single or multiple screw extruder, dynamic or static mixer, colloid mill, homogenizer, sonolator, three roll mill, or a combination thereof, preferably with a three-roll mill.

The amount of the solvent or emollient (B) added in steps (a) and/or (c) can be suitably such that the resulting composition used as crosslinked polyester component (A) comprises from about 10% to about 95%, preferably about 60% to about 90% or about 30% to about 60% weight by weight of the solvent or emollient (B).

### 2. Ratio of Components

In an aspect, the preparation of the elastomer comprises a defined ratio of poly-carboxylic acid, poly-carboxylic acid ester or a combination thereof (i) to polyol (ii), a defined ratio of monocarboxylic acid (iii)(a) to polyol (ii), and a defined ratio of mono-alcohol (iii)(b) to poly-carboxylic acid, poly-carboxylic acid ester, or a combination thereof (i). It has been found that the ratio of (i)/(iii)(a), (ii)/(iii)(a), and (iii)(b)/(i) influences the performance of the polyester elastomer and the performance of the gel made from the polyester elastomer.

In an aspect, the molar ratio of carboxyl functional groups (—COOH) from monocarboxylic acid (iii)(a) to hydroxyl functional groups (—OH) from polyol (ii) is from about 1:2 to about 1:16. In an aspect, the molar ratio of carboxyl functional groups (—COOH) from mono-carboxylic acid (iii)(a) to hydroxyl functional groups (—OH) from polyol (ii) is from about 1:2 to about 1:14. In an aspect, the molar ratio of carboxyl functional groups (—COOH) from mono-carboxylic acid (iii)(a) to hydroxyl functional groups (—OH) from polyol (ii) is from about 1:2 to about 1:10. In an aspect, the molar ratio of carboxyl functional groups (—COOH) from mono-carboxylic acid (iii)(a) to hydroxyl functional groups (—OH) from polyol (ii) is from about 1:2 to about 1:8. In an aspect, the molar ratio of carboxyl functional groups (—COOH) from mono-carboxylic acid (iii)(a) to hydroxyl functional groups (—OH) from polyol (ii) is from about 1:2 to about 1:5. In an aspect, the molar ratio of carboxyl functional groups (—COOH) from mono-carboxylic acid (iii)(a) to hydroxyl functional groups (—OH) from polyol (ii) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid, poly-carboxylic acid ester or a combination thereof (i) to hydroxyl functional groups (—OH) from polyol (ii) is from about 2:1 to about 1:4. In an aspect, the ratio of carboxyl functional groups (—COOH) from poly-carboxylic acid, poly-carboxylic acid ester or a combination thereof (i) to hydroxyl functional groups (—OH) from polyol (ii) is from about 2:1 to about 1:2. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.5. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.25. In an aspect, the ratio of carboxyl functional groups (—COOH) poly-carboxylic acid, poly-carboxylic acid ester or a combination thereof (i) to hydroxyl functional groups (—OH) from polyol (ii) is about 1:2, about 2:3, about 1:1, about 2:1, about 1:4, or about 1:3.

In an aspect, the molar ratio of hydroxyl functional groups (—OH) from mono-alcohol (iii)(b) to carboxyl functional groups (—COOH) from poly-carboxylic acid (i) is from about 1:2 to about 1:16. In an aspect, the molar ratio of hydroxyl functional groups (—OH) from mono-alcohol (iii)(b) to carboxyl functional groups (—COOH) from poly-carboxylic acid (i) is from about 1:2 to about 1:14. In an aspect, the molar ratio of hydroxyl functional groups (—OH) from mono-alcohol (iii)(b) to carboxyl functional groups (—COOH) from poly-carboxylic acid (i) is from about 1:2 to about 1:10. In an aspect, the molar ratio of hydroxyl functional groups (—OH) from mono-alcohol (iii)(b) to carboxyl functional groups (—COOH) from poly-carboxylic acid (i) is from about 1:2 to about 1:8. In an aspect, the molar ratio of hydroxyl functional groups (—OH) from mono-alcohol (iii)(b) to carboxyl functional groups (—COOH) from poly-carboxylic acid (i) is from about 1:2 to about 1:5. In an aspect, the molar ratio of hydroxyl functional groups (—OH) from mono-alcohol (iii)(b) carboxyl functional groups (—COOH) from poly-carboxylic acid (A) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the conversion of carboxyl functional groups (—COOH) to ester functional groups (—CO(O)—) is no less than 80% by mole. The percent conversion is calculated by titration of carboxylic acid functional groups (—COOH) with 0.1N KOH in isopropanol.

In an aspect, esterification is carried out in the absence of a solvent or emollient.

In an aspect, esterification is conducted in solvent or emollient. In another aspect, esterification is conducted in more than one solvent or emollient.

In an aspect, the percentage of solvent or emollient in total raw materials of the esterification reaction is in the range of 0% to 80% by weight. In an aspect, the percentage of solvent or emollient in total raw materials of the esterification reaction is in the range of 0% to 60% by weight. In an aspect, the percentage of solvent or emollient in total raw materials of the esterification reaction is in the range of 0% to 50% by weight. In an aspect, the percentage of solvent or emollient in total raw materials of the esterification reaction is in the range of 0% to 40% by weight. In an aspect, the percentage of solvent or emollient in total raw materials of the esterification reaction is in the range of 0% to 30% by weight. In an aspect, the percentage of solvent or emollient in total raw materials of the esterification is in the range of 0% to 20% by weight. In an aspect, the percentage of solvent or emollient in total raw materials of the esterification reaction is in the range of 0% to 10% by weight. In an aspect, the percentage of solvent or emollient in total raw materials of the esterification reaction is in the range of 10% to 20% by weight. In an aspect, the percentage of solvent or emollient in total raw materials of the esterification reaction is in the range of 20% to 30% by weight.

### 3. Activating Agent

In an aspect, the preparation of the elastomer further comprises an activating agent. In an aspect, the preparation of the elastomer does not comprise an activating agent.

In an aspect, the activating agent is selected from the group consisting of dimethyl dicarbonate, diethyl dicarbonate, dipropyl dicarbonate, di-tertiary-butyl dicarbonate, and combinations thereof.

### 4. Catalyst

In an aspect, the preparation of the elastomer further comprises a catalyst. In an aspect, the preparation of the elastomer does not comprise a catalyst. However, it has been found that when no catalyst is used the reaction times are protracted.

In an aspect, the catalyst is selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth(III) neodecanoate, bismuth(III) citrate, bismuth(III) chloride, bismuth(III) acetate, bismuth(III) phosphate, tin chloride, tin-pyrone, dibutyltin dilaurate, di-n butyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In an aspect, the catalyst is p-toluenesulfonic acid, methanesulfonic acid, phosphoric acid, bismuth neodecanoate, or a combination thereof. In an aspect, the catalyst is methanesulfonic acid.

In an aspect, the catalyst is a salt. In an aspect, the catalyst is a salt selected from the group consisting of Yb(OTf)₃, Sc(OTf)₃, Hf(OTf)₄, Bi(OTf)₃, Al(OTf)₃, Zn(OTf)₂, Mg(ClO₄)₂, Cu(OTf)₂, Ti(OCH(CH₃)₂)₄, and combinations thereof.

### 5. Temperature

In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol with mixing at a pre-determined temperature until an elastomer is formed. In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol, optionally at least one solvent or emollient, and optionally a catalyst with mixing at a pre-determined temperature until an elastomer is formed. In an aspect, the temperature range is from 30° C. to 250° C.

In an aspect, the reaction occurs at a temperature from about 30° C. to about 250° C. In an aspect, the reaction occurs at a temperature from about 60° C. to about 250° C. In an aspect, the reaction occurs at a temperature from about 30° C. to about 125° C. or about 40° C. to about 100° C. In an aspect, the reaction occurs at a temperature of about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C.

### 6. Time

In an aspect, the reaction time is from about 12 hours to about 150 hours. In an aspect, the reaction time is from about 6 hours to about 24 hours. In an aspect, the reaction time is from about 8 hours to about 27 hours. In an aspect, the reaction time is about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 135 hours, about 140 hours, about 145 hours, or about 150 hours.

The reaction time can be adjusted by determination of the gel fraction achieved, and preferably the reaction time is such that the gel fraction of the polyester elastomer is greater than 60%. A method to measure the gel fraction in the polyester elastomer is described below.

### 7. By-Product Removal

In an aspect, the method further comprises removing water and alcohol by-product from the reaction. In a further aspect, the water and alcohol by-products are removed from the reaction by mixing and heating the reaction. In an aspect, the reaction is heated to above about 120° C. to remove the water and alcohol by-products. In an aspect, the water and alcohol by-products are removed from the reaction by nitrogen flow, by vacuum, or a combination thereof. In an aspect, water is removed by nitrogen stripping and vacuum, which have an impact on the reaction time.

### 8. Solvent or Emollient

Solvents or emollients that can be used to prepare the polyester elastomer composition (A) are as described below in the section “Optionally at least one cosmetically acceptable carrier (B)” and can be selected from the solvents or emollients as defined herein. In another aspect, the polyester elastomer in polyester elastomer composition (A) used is in the range of 5 wt % to 100 wt %. In another aspect, the polyester elastomer in polyester elastomer composition (A) used is in the range of 5 wt % to 70 wt %. In another aspect, the polyester elastomer in polyester elastomer composition (A) used is in the range of 10 wt % to 60 wt %. In another aspect, the polyester elastomer in polyester elastomer composition (A) used is in the range of 20 wt % to 50 wt %.

In another aspect, the crosslinked polyester in the polyester elastomer composition or component (A) used is in the range of 50 wt % to 80 wt %. In another aspect, the crosslinked polyester in polyester elastomer composition (A) used is in the range of 50 wt % to 70 wt %. In another aspect, the crosslinked polyester in polyester elastomer composition (A) used is in the range of 60 wt % to 80 wt %. Solvents or emollients that can be used to prepare the polyester elastomer composition are described herein and can be selected from the solvents or emollients as defined herein.

In an aspect, polyester elastomer (A) used is only comprised of crosslinked polyester without solvent or emollient. In an aspect, polyester elastomer (A) used is comprised of polyester without solvent or emollient. In an aspect, polyester elastomer (A) used is comprised of crosslinked polyester with solvent or emollient. In an aspect, polyester elastomer (A) used is comprised of polyester with solvent or emollient.

In an aspect, a defined amount of solvent or emollient (B) is used in the preparation of the polyester elastomer. In an aspect, the amount of solvent is from 0% to 70% of the total weight of poly-carboxylic acid (i), polyol (ii), mono-carboxylic acid (iii)(a) and mono-alcohol (iii)(b). In an aspect, the amount of solvent is from 0% to 50% of the total weight of poly-carboxylic acid (i), polyol (ii), mono-carboxylic acid (iii)(a) and mono-alcohol (iii)(b). In an aspect, the amount of solvent is from 0% to 40% of the total weight of poly-carboxylic acid (i), polyol (ii), mono-carboxylic acid (iii)(a) and mono-alcohol (iii)(b). In an aspect, the amount of solvent is from 0% to 30% of the total weight poly-carboxylic acid (i), polyol (ii), mono-carboxylic acid (iii)(a) and mono-alcohol (iii)(b). In an aspect, the range of solvent is from 0% to 20% of the total weight of poly-carboxylic acid (i), polyol (ii), mono-carboxylic acid (iii)(a) and mono-alcohol (iii)(b). In an aspect, the amount of solvent is from 10% to 50% of the total weight of poly-carboxylic acid (i), polyol (ii), mono-carboxylic acid (iii)(a) and mono-alcohol (iii)(b). In an aspect, the amount solvent is 50%, 40%, 30%, 20%, or 10% of the total weight of poly-carboxylic acid (i), polyol (ii), mono-carboxylic acid (iii)(a) and mono-alcohol (iii)(b).

In an aspect, the amount of solvent or emollient (B) is from 0% to 30% of the total weight of poly-carboxylic acid (i) and mono-carboxylic acid (iii)(a). In an aspect, the amount of solvent is from 0% to 20% of the total weight of poly-carboxylic acid (i) and mono-carboxylic acid (iii)(a). In an aspect, the amount of solvent is from 0% to 10% of the total weight of poly-carboxylic acid (i) and mono-carboxylic acid (iii)(a). In an aspect, the amount of solvent is from 10% to 30% of the total weight of poly-carboxylic acid (i) and mono-carboxylic acid (iii)(a). In an aspect, the solvent is 30%, 20%, or 10% of the total weight of poly-carboxylic acid (i) and mono-carboxylic acid (iii)(a).

In an aspect, no solvent or emollient (B) is used to prepare the elastomer (A).

In an aspect, a solvent or emollient (B) is used to prepare the polyester elastomer (A), which is removed after preparing the polyester elastomer (A) to form a polyester elastomer (A) powder. In an aspect, to the polyester elastomer powder a solvent/emollient (B) can be added again to form a polyester elastomer (A) gel, optionally applying a shear force as described below.

In an aspect, polyester elastomer (A) is made from C36 dimer acid, diglycerol, and isostearic acid with about 10% to about 40% by weight squalane based on the total weight of the polyester elastomer (A) and the squalane. That is, in an aspect the invention relates to a composition of a polyester elastomer made from C36 dimer acid, diglycerol, isostearic acid, and squalane, comprising about 10% to about 40% by weight squalane.

In an aspect, polyester elastomer (A) is made from hydrogenated C36 dimer acid, diglycerol, and oleic acid without any solvent or emollient (B).

### 9. Powder or Gel

As mentioned above in an aspect, the polyester elastomer (A) is provided as a (at room temperature (23° C.) flowable or fluid) gel or a (dry) powder.

In an aspect, the polyester elastomer (A) is processed into a powder.

In an aspect, the polyester elastomer (A) is processed into a gel as described herein.

In an aspect, the polyester elastomer composition (A) used comprises at least one solvent or emollient added to the polyester elastomer during the shearing force process to form a gel. Solvents or emollients (B) that can be used to prepare the polyester elastomer composition are described herein in the section “Optionally at least one solvent or emollient (B)” and can be selected from the solvents as defined herein. In an aspect, the solvent or emollient is from about 20% to about 95% weight by weight of the composition. In an aspect, the solvent or emollient is from about 20% to about 50% weight by weight of the composition. In an aspect, the solvent or emollient is from about 50% to about 90% weight by weight of the composition. In an aspect, the solvent or emollient is from about 70% to about 90% weight by weight of the composition. In an aspect, the polyester elastomer composition (A) comprises from about 50% to about 90% weight by weight of solvent or emollient, from about 50% to about 80% weight by weight of solvent or emollient, from about 50% to about 70% weight by weight of solvent or emollient, or from about 50% to about 60% weight by weight of solvent or emollient. In some embodiments, the polyester elastomer composition (A) comprises about 20%, about 30%, about 40%, about 50%, about 60%, about 70%, about 80%, or about 90% weight by weight of solvent or emollient.

### D. Optionally at Least One Solvent or Emollient (B)

In an aspect the solvent or emollient (B) is preferably selected from the emollients or solvents as described herein below. The solvent or emollient (B) can be present in the preparation of the crosslinked polyester component (A) and/or can be added after the preparation of the crosslinked polyester component (A).

In an aspect, the preparation of the polyester elastomer (A) can occur in the presence of a solvent. The solvent can also act as an emollient, preferably a cosmetic emollient. When the solvent acts also as an emollient it also provides a softening, protecting, moisturizing, and/or lubricating effect to the skin. In an aspect, the solvent or emollient is preferably a biobased or naturally derived. In an aspect, the solvent or emollient is a triglyceride solvent, a mono-ester solvent, a di-ester solvent, a citrate ester solvent, an ether solvent, a carbonate solvent, a hydrocarbon solvent, a silicone solvent, or a combination thereof.

In an aspect, the solvent is a triglyceride solvent of formula (XIV)

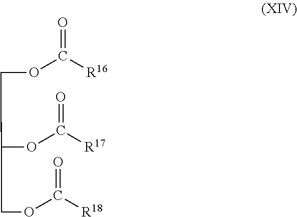

wherein
each R¹⁶, R¹⁷, and R¹⁸ are independently C1-C35 alkyl group, C1-C35 heteroalkyl group, C2-C35 alkene group, or C2-C35 heteroalkene group.

In an aspect, the solvent is of formula (XIV), wherein R¹⁶, R¹⁷, and R¹⁸ are independently C2-C17 alkyl group or C2-C17 alkylene group.

In an aspect, the solvent is a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride solvent is selected from the group consisting of caprylic/capric triglyceride, triheptanoin, and combinations thereof.

In an aspect, the solvent is a mono-ester solvent of formula (XV)

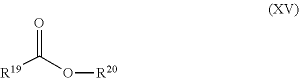

wherein
R¹⁹ is C1-C35 alkyl group, C1-C35 heteroalkyl group, C2-C35 alkene group, or C2-C35 heteroalkene group; and
R²⁰ is C1-C35 alkyl group, C1-C35 heteroalkyl group, C2-C35 alkene group, or C2-C35 heteroalkene group.

In an aspect, the solvent is a mono-ester solvent of formula (XV), wherein R¹⁹ is C5-C17 alkyl group or C5-C17 alkene group and R²⁰ is C2-C17 alkyl group or C2-C17 alkene group.

In an aspect, the solvent is a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C12-C13 alkyl lactate, C12-C15 alkyl lactate, isostearyl lactate, glycereth-5-lactate, lauryl lactate, myristyl lactate, oleyl lactate, laureth-2-benzoate, C12-C15 alkyl benzoate, C12-C15 pareth-3-benzoate, dipropylene glycol benzoate, isodecyl salicylate, C12-C15 alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, myristyl neopentanoate, isostearyl behenate, octyldodecyl neopentanoate, tridecyl neopentanoate, and combinations thereof. In an aspect, the mono-ester solvent is selected from the group consisting of cococaprylate/caprate, coco-caprylate, jojoba oil, isoamyl laurate, methylheptyl isostearate, C12-C13 alkyl lactate, C12-C15 alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In an aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent is:
(a) a di-ester solvent of formula (XVI)

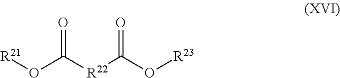

wherein
   R²¹ is C1-C35 alkyl group, C1-C35 heteroalkyl group, C2-C35 alkene group, or C2-C35 heteroalkene group; and
   R²² and R² are independently C1-C35 alkyl group, C1-C35 heteroalkyl group, C2-C35 alkene group, or C2-C35 heteroalkene group; or
(b) a di-ester solvent of formula (XVII)

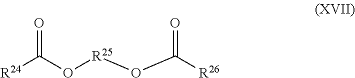

wherein
   R²⁴ is C1-C35 alkyl group, C1-C35 heteroalkyl group, C2-C35 alkene group, or C2-C35 heteroalkene group; and
   R²⁵ and R² are independently H, C1-C35 alkyl group, C1-C35 heteroalkyl group, C2-C35 alkene group, or C2-C35 heteroalkene group; or
(c) a di-ester solvent of formula (XVIII)

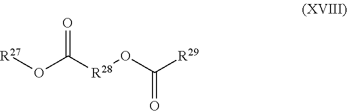

wherein
   R²⁷ and R²⁸ are independently C1-C35 alkyl group, C1-C35 heteroalkyl group, C2-C35 alkene group, or C2-C35 heteroalkene group; and
   R²⁹ is H, C1-C35 alkyl group, C1-C35 heteroalkyl group, C2-C35 alkene group, or C2-C35 heteroalkene group.

In an aspect, the solvent is a di-ester solvent of formula (XVI), formula (XVII), or formula (XVIII), wherein R²³, R²⁶, and R²⁹ is C2-C10 alkyl group or C2-C10 alkene group and R²¹, R²⁴, R²⁷, and R²², R²⁵, and R²⁸ are independently C1-C12 alkyl group or C2-C12 alkene group.

In an aspect, the di-ester solvent is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In an aspect, the di-ester solvent is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent is a citrate ester solvent of formula (XVIII)

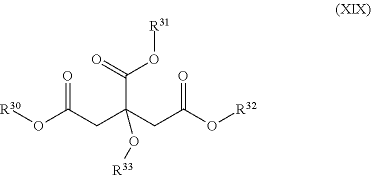

wherein
R³⁰, R³¹, R³², and R³³ are independently H, C1-C35 alkyl group, C1-C35 heteroalkyl group, C2-C35 alkene group, or C2-C35 heteroalkene group, wherein at least one of R³⁰, R³¹, R³², and R³³ is not H.

In an aspect, the solvent is a citrate ester solvent of formula (XVIII), wherein R³⁰, R³¹, and R³² are independently C1-C10 alkyl group or C2-C10 alkene group and R³³ is an acetyl group.

In an aspect, the solvent is a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent is an ether solvent of formula (XX)

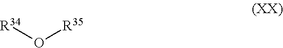

wherein
R³⁴ and R³⁵ are independently H, C2-C20 alkyl group, C2-C20 heteroalkyl group, C2-C20 alkene group, or C2-C20 heteroalkene group, wherein at least one of R³⁴ and R is not H.

In an aspect, the solvent is an ether solvent of formula (XIX), wherein R³⁴ and R³⁵ are independently C2-C20 alkyl group.

In an aspect, the solvent is an ether solvent selected from the group consisting of dicaprylyl ether, didecyl ether, panthenyl ethyl ether, dicetyl ether, dimyristyl ether, distearyl ether, dilauryl ether, and combinations thereof. In an aspect, the ether solvent is selected from the group consisting of dicaprylyl ether, didecyl ether, and combinations thereof.

In an aspect, the solvent is a carbonate solvent of formula (XXI)

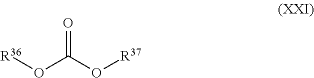

wherein
R³⁶ and R³⁷ are independently H, C2-C20 alkyl group, C2-C20 heteroalkyl group, C2-C20 alkene group, or C2-C20 heteroalkene group.

In an aspect, the solvent is a carbonate solvent of formula (XX), wherein R³⁶ and R³⁷ are independently C2-C20 alkyl group.

In an aspect, the solvent is a carbonate solvent selected from the group consisting of dicaprylyl carbonate, diethyl hexyl carbonate, and combinations thereof.

In an aspect, the solvent is a hydrocarbon with number of carbon atoms from C4 to C60. In an aspect, the solvent is a hydrocarbon with a number of carbon atoms from C10 to C50. In an aspect, the solvent is a hydrocarbon with a number of carbon atoms from C20 to C40.

In an aspect, the solvent is a hydrocarbon solvent selected from the group consisting of farnesene, hydrogenated farnesene, coconut alkanes, coconut/palm kernel alkanes, C9-C12 alkane, C10-C13 alkane, C12-C17 alkane, C13-C14 alkane, C13-C15 alkane, C14-C17 alkane, C14-C19 alkane, C14-C20 alkane, C14-C22 alkane, C15-C19 alkane, C21-C28 alkane, C17-C23 alkane, C9-C12 isoalkane, C9-C13 isoalkane, C9-C14 isoalkane, C9-C16 isoalkane, C10-C11 isoalkane, C10-C12 isoalkane, C10-C13 isoalkane, C11-C12 isoalkane, C11-C13 isoalkane, C11-C14 isoalkane, C12-C14 isoalkane, C12-C15 isoalkane, C12-C20 isoalkane, C13-C14 isoalkane, C13-C16 isoalkane, C14-C16 isoalkane, C15-C19 isoalkane, C10-C16 olefin, C12-C18 olefin, C18-C26 olefin, C20 olefin, C20-C24 olefin, C24-C30 olefin, C26-C28 olefin, C26-C54 olefin, C28-C36 olefin, C28-C52 olefin, C30-C38 olefin, C30-C45 olefin, C4-C12 olefin, C4-C6 olefin, C5-C6 olefin, hydrogenated poly(C6/C10/C14 olefin), hydrogenated poly(C6-C12 olefin), hydrogenated poly(C6-C14 olefin), hydrogenated poly(C6-C20 olefin), hydrogenated poly(C8/C12 olefin), poly(C20-C28 olefin), poly(C30-C45 olefin), poly(C4-C12 olefin), poly(C6-C14 olefin), hexadecene, C32 alkane, C32 isoalkane, C54 alkane, C54 isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon solvent is selected from the group consisting of squalane, farnesene, hydrogenated farnesene, coconut alkanes, C9-C12 alkane, C13-C15 alkane, C14-C19 alkane, C14-C20 alkane, C14-C22 alkane, C15-C19 alkane, C13-C16 isoalkane, dodecane, undecane, tridecane, tetradecane, pentadecane, hexadecane, hexadecene, octadecane, squalane, isododecane, isohexadecane, C32 alkane, C32 isoalkane, C54 alkane, C54 isoalkane, and combinations thereof. In an aspect, the hydrocarbon solvent is selected from the group consisting of squalane, hydrogenated farnesene, coconut alkanes, C9-C12 alkane, C13-C15 alkane, C13-C16 isoalkane, C14-C19 alkane, dodecane, tetradecane, isododecane, hexadecane, octadecane, hexadecene, C32 alkane, C32 isoalkane, C54 alkane, C54 isoalkane, and combinations thereof.

In an aspect, the hydrocarbon solvent is selected from the group consisting of squalane, C32 alkane, C32 isoalkane, C54 alkane, C54 isoalkane, and combinations thereof.

In an aspect, the solvent may be a chemical UV filter. The UV filter solvent may be selected from the group consisting of ethyl dimethyl PABA, ethylhexyl methoxycinnamate, ethylhexyl salicylate, homosalate, isoamyl p-Methoxycinnamate, menthyl anthranilate, octocrylene, polysilicone-15, terephthalylidene dicamphor sulfonic acid, triethanolamine salicylate, and combinations thereof.

In an aspect, the solvent is a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

E. Optionally One or More Other Emulsifier or Emulsion Stabilizer Used in the Cosmetic Field, Different from Components (A) or (B).

In an aspect the emulsifier or emulsion stabilizer (C) is preferably selected from the emulsifier or emulsion stabilizer as described herein below. The emulsifier or emulsion stabilizer (C) can be present in the preparation of the crosslinked polyester component (A) and/or can be added after the preparation of the crosslinked polyester component (A). In an aspect, the emulsifier or emulsion stabilizer (C) is mixed with crosslinked polyester component (A) before used in personal care formulation as a mixture. In an aspect, the emulsifier or emulsion stabilizer (C) is mixed with crosslinked polyester component (A) and solvent or emollient (B) before used in personal care formulation as a mixture. In an aspect, the emulsifier or emulsion stabilizer (C) is used in formulation as a separate phase from crosslinked polyester component (A) and solvent or emollient (B).

In an aspect the emulsifiers or emulsion stabilizers (C), different from components (A) or (B) are suitably selected from the group consisting of
(i) Non-Ionic Emulsifiers and Surfactants, preferably selected from the group consisting of Polysorbates (e.g., Polysorbate 20, Polysorbate 60, Polysorbate 80), Cetearyl Alcohol, Ceteareth-20, Glyceryl Stearate and PEG-Stearates, such as PEG-100 Stearate, Sodium Stearoyl Lactylate, PEG-7 glyceryl cocoate, mixtures of Cetearyl Alcohol and Cetearyl Glucoside (e.g. Montanov™ 68), and PEG-Hydrogenated Castor Oils (e.g. PEG-40 Hydrogenated Castor Oil);
(ii) Anionic Emulsifiers and Surfactants preferably selected from the group consisting of Sodium Lauryl Sulfate (SLS), Sodium Coco-Sulfate, Sodium Stearate, Disodium Laureth Sulfosuccinate, Ammonium Lauryl Sulfate, Sodium Cocoyl Isethionate, Sodium lauroyl sarcosinate, Sodium Myreth Sulfate, Sodium Lauryl Glutamate, and Sodium Olefin Sulfonate;
(iii) Cationic Emulsifiers and Surfactants preferably selected from the group consisting of Behentrimonium Chloride, Cetrimonium Bromide, and Stearalkonium Chloride;
(iv) Amphoteric Emulsifiers and Surfactants preferably selected from the group consisting of Cocamidopropyl Betaine, Lauramidopropyl Betaine, Disodium Cocoamphodiacetate, and Cocamidopropyl Hydroxysultaine;
(v) Fatty Acid Derivatives preferably selected from the group consisting of Stearic Acid, Oleic Acid, Palmitic Acid, and Lauric Acid;
(vi) Emulsifiers and Surfactants different from the aforementioned Emulsifiers and Surfactants (i)-(v) preferably selected from the group consisting of Polyethylene Glycol (PEG) Esters, e.g., PEG-40 Castor Oil, PEG-100 Stearate;
   and combinations of the aforementioned Emulsifiers and Surfactants (i)-(vi).

In an aspect the emulsifiers or emulsion stabilizers (C) is phospholipids such as lecithin, hydrogenated lecithin and mixtures thereof.

In an aspect the emulsifiers or emulsion stabilizers (C) is selected from glyceryl esters which are obtainable by reacting glycerol with 1 to 3 monocarboxylic acid having from 4 to 24 carbon atoms. The monocarboxylic acid is represented by the formula Rₐ—COOH and Rₐ is commonly a C3 to C23 aliphatic hydrocarbyl group. Desirably, Rₐ is a C5 to C21, preferably C7 to C19, alkyl, alkenyl or alkadienyl group. Rₐ may be straight chain or branched and saturated or unsaturated. Examples of the monocarboxylic acid include valeric, caproic, enanthic, capric, caprylic, lauric, myristic, palmitic, stearic, iso-stearic, arachidic, behenic, oleic, linoleic, palmitoleic acid and mixtures thereof. Preferably the monocarboxylic acid is saturated. Examples of the glyceryl esters include Glyceryl Caprylate, Glyceryl Oleate, Glyceryl Stearate SE, Glyceryl Stearate, Glyceryl Laurate, Glyceryl Hydroxystearate, Glyceryl Heptanoate, Glyceryl Dilaurate, Glyceryl Olivate, Glyceryl Undecylenate, Glyceryl Isostearate and mixtures thereof.

In an aspect, the emulsifier or emulsion stabilizers (C) is selected from glyceryl esters which are obtainable by reacting glycerol with 1 to 3 hydroxyl carboxylic acid having from 4 to 24 carbon atoms, one or more hydroxyl groups, and one or more carboxylic acid groups. Examples of the hydroxyl carboxylic acid include glycolic acid, hydroxypropionic acids, lactic acid, hydroxybutyric acids, citric acid, salicylic acid, ricinoleic acid, hydroxystearate acid and mixtures thereof. Examples of the glyceryl esters include Glyceryl Hydroxystearate, Glyceryl Stearate Citrate, Glyceryl Oleate Citrate and mixtures thereof.

In an aspect the emulsifier or emulsion stabilizers (C) is selected from diglyceryl esters which are obtainable by reacting diglycerol with 1 to 4 monocarboxylic acid having from 4 to 24 carbon atoms. The monocarboxylic acid is represented by the formula R_{b}—COOH and R_{b} is commonly a C3 to C23 aliphatic hydrocarbyl group. Desirably, R_{b} is a C5 to C21, preferably C7 to C19, alkyl, alkenyl or alkadienyl group. R_{b} may be straight chain or branched and saturated or unsaturated. Examples of the monocarboxylic acid include valeric, caproic, enanthic, capric, caprylic, lauric, myristic, palmitic, stearic, iso-stearic, arachidic, behenic, oleic, linoleic, palmitoleic acid and mixtures thereof. Preferably the monocarboxylic acid is saturated. Examples of the diglyceryl esters include Polyglyceryl-2 Isostearate, Polyglyceryl-2 Stearate, Polyglyceryl-2 Sesquicaprylate, Polyglyceryl-2 Sesquioleate, Polyglyceryl-2 Sesquiisostearate, Polyglyceryl-2 Distearate and mixtures thereof.

In an aspect, the emulsifier or emulsion stabilizers (C) is selected from diglyceryl esters which are obtainable by reacting glycerol with 1 to 10 hydroxyl carboxylic acid having from 4 to 24 carbon atoms, one or more hydroxyl groups, and one or more carboxylic acid groups. Examples of the hydroxyl carboxylic acid include glycolic acid, hydroxypropionic acids, lactic acid, hydroxybutyric acids, citric acid, salicylic acid, ricinoleic acid, hydroxystearate acid and mixtures thereof. Examples of the polyglyceryl esters include polyglyceryl-2 dipolyhydroxystearate.

In an aspect the emulsifier or emulsion stabilizers (C) is selected from polyglyceryl esters which are obtainable by reacting polyglycerol having from 3 to 20 glycerol units with a 1 to 10 monocarboxylic acid having from 4 to 24 carbon atoms. The monocarboxylic acid is represented by the formula R_{c}—COOH and R_{c} is commonly a C3 to C23 aliphatic hydrocarbyl group. Desirably, R_{c} is a C5 to C21, preferably C7 to C19, alkyl, alkenyl or alkadienyl group. R_{c} may be straight chain or branched and saturated or unsaturated. Examples of the monocarboxylic acid include valeric, caproic, enanthic, capric, caprylic, lauric, myristic, palmitic, stearic, iso-stearic, arachidic, behenic, oleic, linoleic, palmitoleic acid and mixtures thereof. Preferably the monocarboxylic acid is saturated. Examples of the polyglyceryl esters include Polyglyceryl-3 Caprate, Polyglyceryl-3 Laurate, Polyglyceryl-3 Stearate, Polyglyceryl-3 Oleate, Polyglyceryl-3 Diisostearate, Polyglyceryl-3 Triolivate, Polyglyceryl-4 Caprate, Polyglyceryl-4 Isostearate, Polyglyceryl-6 Laurate, Polyglyceryl-6 Dioleate, Polyglyceryl-6 Distearate, Polyglyceryl-6 Octastearate, Polyglyceryl-10 Caprylate/Caprate, Polyglyceryl-10 Laurate, Polyglyceryl-10 Myristate, Polyglyceryl-10 Stearate, Polyglyceryl-10 Diisostearate, Polyglyceryl-10 Decaoleate and mixtures thereof.

In an aspect the emulsifier or emulsion stabilizers (C) is selected from polyglyceryl esters which are obtainable by reacting polyglycerol having from 3 to 20 glycerol units with 1 to 10 hydroxyl carboxylic acid having from 4 to 24 carbon atoms, one or more hydroxyl groups, and one or more carboxylic acid groups. Examples of the hydroxyl carboxylic acid include glycolic acid, hydroxypropionic acids, lactic acid, hydroxybutyric acids, citric acid, salicylic acid, ricinoleic acid, hydroxystearate acid and mixtures thereof. Examples of the polyglyceryl esters include Polyglyceryl-3 Dicitrate/Stearate, polyglyceryl-3 polyricinoleate, polyglyceryl-6 polyricinoleate, polyglyceryl-4 diisostearate/polyhydroxystearate/sebacate, and mixtures thereof.

In an aspect the emulsifier or emulsion stabilizers (C) is selected from sorbitan or sorbitol ester which is obtainable by reacting sorbitan or sorbitol with 1 to 3 monocarboxylic acids having from 4 to 24 carbon atoms. The monocarboxylic acid is represented by the formula R_{d}—COOH and R_{d} is commonly a C3 to C23 aliphatic hydrocarbyl group. Desirably, R_{d} is a C5 to C21, preferably C7 to C19, alkyl, alkenyl or alkadienyl group. R_{d} may be straight chain or branched and saturated or unsaturated. Examples of the monocarboxylic acid include valeric, caproic, enanthic, capric, caprylic, lauric, myristic, palmitic, stearic, iso-stearic, arachidic, behenic, oleic, linoleic, palmitoleic acid and mixtures thereof. Preferably the monocarboxylic acid is saturated. Examples of the sorbitan or sorbitol esters include Sorbitan Caprylate, Sorbitan Sesquicaprylate, Sorbitan Laurate, Sorbitan Cocoate, Sorbitan Palmitate, Sorbitan Oleate, Sorbitan Stearate, Sorbitan Isostearate, Sorbitan Olivate, Sorbitan Sesquioleate, Sorbitan Trioleate, Sorbitan Tristearate and mixtures thereof.

In an aspect the emulsifier or emulsion stabilizers (C) is selected from sugar ester from mono- or di-saccharides with 1 to 6 monocarboxylic acid having from 4 to 24 carbon atoms. The monocarboxylic acid is represented by the formula Rₑ—COOH and Rₑ is commonly a C3 to C23 aliphatic hydrocarbyl group. Desirably, Rₑ is a C5 to C21, preferably C7 to C19, alkyl, alkenyl or alkadienyl group. Rₑ may be straight chain or branched and saturated or unsaturated. Examples of the monocarboxylic acid include valeric, caproic, enanthic, capric, caprylic, lauric, myristic, palmitic, stearic, iso-stearic, arachidic, behenic, oleic, linoleic, palmitoleic acid and mixtures thereof. Preferably the monocarboxylic acid is saturated. Examples of the sugar esters include Methyl Glucose Sesquistearate, Methyl Glucose Dioleate, Sucrose Cocoate, Sucrose Palmitate, Sucrose Stearate, Sucrose Distearate, Sucrose Polystearate and mixtures thereof.

In an aspect the emulsifier or emulsion stabilizers (C) is selected from glycolipids such as sophorolipids and rhamnolipids.

In an aspect the emulsifier or emulsion stabilizers (C) is selected from amino acid derivatives. Examples include Sodium Myristoyl Glutamate, Sodium Cocoyl Glutamate, Sodium Lauroyl Glutamate, Sodium Stearoyl Glutamate and mixtures thereof.

In an aspect the emulsifier or emulsion stabilizers (C) is selected from alkyl polyglycosides (APGs) produced by combining a sugar such as glucose with a fatty alcohol in the presence of acid catalysts at elevated temperatures. Examples of alkyl polyglycosides (APGs) include Caprylyl/Capryl Glucoside, Cetearyl Glucoside, Decyl Glucoside, Lauryl Glucoside, Coco-Glucoside and mixtures thereof.

In an aspect the emulsifier or emulsion stabilizers (C) is selected from polyester or oligoester which is obtainable by reacting glycerol, diglycerol or polyglycerol having from 3 to 20 glycerol units with a dicarboxylic acid or cyclic anhydride of such dicarboxylic acid having from 4 to 22 carbon atoms and a monocarboxylic acid having from 4 to 24 carbon atoms. The monocarboxylic acid is represented by the formula R_{f}—COOH and R1 is commonly a C3 to C23 aliphatic hydrocarbyl group. Desirably, R_{f} is a C5 to C21, preferably C7 to C19, alkyl, alkenyl or alkadienyl group. R_{f} may be straight chain or branched and saturated or unsaturated. Examples of this kind of polyester and oligoester include polyglyceryl-4 diisostearate/polyhydroxystearate/sebacate, polyglyceryl-4 laurate/succinate, diisostearoyl polyglyceryl-3 dimer dilinoleate, and mixtures thereof.

In an aspect, the preparation of the polyester elastomer (A) can occur in the presence of a solvent. The solvent can also act as an emollient, preferably a cosmetic emollient. When the solvent acts also as an emollient it also provides a softening, protecting, moisturizing, and/or lubricating effect to the skin. In an aspect, the solvent or emollient is preferably a biobased or naturally derived. In an aspect, the solvent or emollient is a triglyceride solvent, a mono-ester solvent, a di-ester solvent, a citrate ester solvent, an ether solvent, a carbonate solvent, a hydrocarbon solvent, a silicone solvent, or a combination thereof.

### Process to Prepare the Polyester Elastomer Component (A) as a Powder

In an aspect, the polyester elastomer obtained by the esterification process is crumbled or processed to form a polyester elastomer powder. In another aspect, the polyester elastomer is processed by a three-roll mill to form a polyester elastomer powder.

The polyester elastomer composition (A) can be also obtained as a powder if it still comprises a solvent or emollient added to the reaction mixture.

### Process to Prepare the Polyester Elastomer Component (A) as a Gel Composition

In an aspect, polyester elastomer composition (A) is prepared by combining the polyester elastomer with one or more solvents or emollients to form a polyester elastomer gel.

In an aspect, polyester elastomer and solvent/emollient mixture are processed with a homogenizer to produce a gel optionally applying a shear force, e.g. by a high-shear disperser mixer.

In an aspect to prepare the polyester elastomer composition (A) as gel, a polyester elastomer is swelled in a solvent or emollient (B) before being processed to make a gel at a temperature under 23° C. In an aspect, the time of polyester elastomer swelling in a solvent or emollient is from 1 hour to 1 week. In an aspect, the elastomer is subject to swelling in a solvent or emollient (B) from 10 minutes to 1 week, from 10 minutes to 4 days, from 10 minutes to 3 days, from 10 minutes to 2 days, from 10 minutes to 1 day, from 10 minutes to 12 hours, from 10 minutes to 6 hours, from 10 minutes to 3 hours, from 10 minutes to 2 hours, from 10 minutes to 1 hour, or from 10 minutes to 30 minutes.

Once the initially produced polyester elastomer is prepared, it can be mixed with an additional quantity of at least one solvent or emollient (B) that can be different from the solvent emollient (B) used to prepare the initially produced elastomer. In some aspects, the at least one solvent or emollient (B) used to prepare the elastomer is the same as the at least one solvent or emollient used to prepare the elastomer composition. The addition of an additional quantity of at least one solvent or emollient dilutes the gel composition and thereby adjusts its viscosity.

The method of preparing the polyester elastomer composition (A) as gel, suitably comprises:
(i) combining a polyester elastomer with at least one solvent or emollient thereby forming a swollen polymer elastomer; and
(ii) subjecting the swollen polyester elastomer to shear force thereby forming a polyester elastomer composition.

In an aspect, the polyester elastomer composition is a powder, a gel, or a paste.

In an aspect, the at least one solvent or emollient (B) is selected from the solvents or emollients described herein.

### Characterization of the Crosslinked Polyester Components (A)

In an aspect, the solubility of polyester elastomer (A) is measured by mixing 1 gram of polyester elastomer with 100 gram of test solvent with magnetic stirring in a sealed glass container for 24 hours under room temperature (23° C.). Afterward the polyester elastomer (A) and test solvent is passed through filtration and the solid on filter is obtained and dried under 80° C. for 20 hours or dried to constant weight, optionally in vacuo. The dried solid is considered as the fraction of polyester elastomer (A) which is not soluble in the test solvent.

In an aspect, the fraction of the polyester elastomer (A) which is not soluble in the ethyl acetate by weight is greater than or equal to 40% of the total weight of the polyester elastomer (A).

In another aspect, the fraction of the polyester elastomer (A) which is not soluble in ethyl acetate is 40% to 90% of the total weight of the polyester elastomer (A). In another aspect, the fraction of the polyester elastomer (A) which is not soluble in ethyl acetate is 50% to 90% of the total weight of the polyester elastomer (A).

A sample of polyester elastomer (A) without solvent or emollient, or a sample of polyester elastomer (A) with solvent or emollient can be used. In case a sample of polyester elastomer (A) with solvent or emollient is used, the weight of the solvent or emollient in the composition is considered and subtracted from the total composition weight to determine the weight percentage of the fraction of the polyester elastomer (A) which is not soluble in the ethyl acetate.

The method of Soxhlet extraction can be used to determine the % by weight of crosslinked polyester (gel fraction) in the polyester elastomer (A). The basis for the test is to extract soluble low Mw components (and optionally present solvents or emollients) from the high Mw and crosslinked insoluble components through an extraction test. The percentage of the crosslinked polyester is defined as the ratio of the weight of insoluble residue (dried gel) to the initial weight of the polyester elastomer sample.

The gel fraction of the polyester elastomers (A), defined as is determined as follows.

$$Gel fraction ( \% ) = 100 \times \frac{weight of dried gel ( insoluble residue of the extraction )}{total weight of the polyester elastomer used in the extraction} .$$

A sample of polyester elastomer without solvent or emollient, or a sample of polyester elastomer with solvent or emollient can be used. In case a sample of polyester elastomer with solvent or emollient is used, the weight of the solvent or emollient in the polyester elastomer is considered and subtracted from the sample weight to determine the total weight of the polyester elastomer (A) used in the extraction in the formula above.

A cellulose extraction thimble is weighed. 3.0-3.5 grams of a sample of a polyester elastomer is weighed and placed in the cellulose extraction thimble. About 125-150 mL of ethyl acetate (EtOAc) are placed in a 250 mL round bottom flask. The thimble with the sample is placed into the Soxhlet extraction column. The EtOAc solution is heated to 80° C. (Reflux, bp 77° C.) and maintained at reflux for 1 hour to extract the polymer sample solubles and the optional present solvent or emollient. After 2 hours the EtOAc solution is allowed to cool to room temperature (about 23° C.). The thimble containing the polymer residue is removed from the Soxhlet apparatus. The thimble is placed in a desiccator under vacuum, a vacuum oven, or a vented oven (50° C.) to remove the residual EtOAc (24 hours). After the EtOAc has been removed, the weight of the thimble containing the dried gel is determined. The weight of the dried gel is calculated by subtraction of the weight of the cellulose extraction thimble. The gel fraction is calculated from the above equation. Any amount of solvent or emollient in the sample is subtracted from the “total weight of the polyester elastomer (A) used in the extraction”.

In an aspect, the gel fraction of the polyester elastomer (A) is greater than 20%. In an aspect, the gel fraction of the polyester elastomer (A) is greater than 40%. In an aspect, the gel fraction of the polyester elastomer (A) is greater than 50%. In an aspect, the gel fraction of the polyester elastomer is greater than 60%. In an aspect, the gel fraction of the polyester elastomer (A) is greater than 70%.

Swelling test for the polyester elastomer (A) is to determine the swelling capacity of the polyester elastomer (A) through a weight of solvent or emollient retained by the polyester elastomer. The swelling ratio (SR—sometimes called swelling value) is determined according to the equation:

$$SR = \frac{Ws - Wi}{Wi}$$

where:
Ws is the weight of the swollen polyester elastomer, and
Wi is the weight of the initial (dry polymer).

The swelling ratio of the polyester elastomer (A) is suitably determined as follows.

A sample of polyester elastomer (A) without solvent or emollient, or a sample of polyester elastomer with solvent or emollient can be used. In case a sample of polyester elastomer (A) with solvent or emollient is used, the weight of the solvent or emollient already present in the composition is considered and subtracted from the initial sample weight Wi.

The swelling procedure is carried out at ambient temperature (23° C.).

About 1.9-2.1 grams of the polyester elastomer is placed in a 25 mL beaker. In the same beaker the polyester elastomer is mixed with 24.9-25.1 grams of coco-caprylate/caprate as solvent. The polyester elastomer is allowed to disperse and absorb (swell) the solvent for 30 minutes. The weight of the filter component (such as Thermo Scientific™ Nalgene™ Rapid-Flow™ Sterile Disposable Filter Units) is determined. After the polyester elastomer has swelled, the mixture in the beaker is mixed and poured into the filter. The beaker is rinsed with about 4.9-5.1 grams of coco-caprylate/caprate solvent to complete transfer of the swelled polyester elastomer. The excess solvent in the gel mixture is allowed to pass through the filter. The filter with swollen polyester elastomer is weighed when no excess solvent is observed on its surface (which can take about 4-18 hours) to give Ws.

The swelling ratio (SR) is calculated by the equation above.

In an aspect, the swelling ratio of the elastomer (A) is from about 1 gram/gram to about 15 gram/gram, from about 1 gram/gram to about 5 gram/gram, from about 1 gram/gram to about 4 gram/gram, from about 1 gram/gram to about 2 gram/gram. In some embodiments, the swelling value of the elastomer is about 15 gram/gram, about 14 gram/gram, about 13 gram/gram, about 12 gram/gram, about 11 gram/gram, about 10 gram/gram, about 9 gram/gram, about 8 gram/gram, about 7 gram/gram, about 6 gram/gram, 5 gram/gram, about 4.8 gram/gram, about 4.6 gram/gram, about 4.4 gram/gram, about 4.2 gram/gram, about 4 gram/gram, about 3.8 gram/gram, about 3.6 gram/gram, about 3.4 gram/gram, about 3.2 gram/gram, about 3 gram/gram, about 2 gram/gram, or about 1 gram/gram.

### Polyester Elastomer Component (A) Used as a Gel

As mentioned before in an aspect, a polyester elastomer composition (A) is prepared by shearing the polyester elastomer with a solvent or emollient, as described herein, to form a sheared polyester elastomer gel. In another aspect, a polyester elastomer gel is prepared by combining the polyester elastomer, as described herein, with a solvent or emollient, as described herein, thereby forming a mixture and shearing the mixture.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, three roll mill, or a combination thereof.

In an aspect, the viscosity of the polyester elastomer (A) used in gel form is from about 10 cp to about 1,000,000 cp as measured by rheometer at a shear rate of 0.1 s-1. In an aspect, the viscosity of the gel at 25° C. is from about 30,000 cp to about 900,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, −40-about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

The viscosity of the polyester elastomer gel is measured by Anton Paar rheometer MCR 301, with probe PP25/S at gap 1 mm. The measuring profile is to use flow curve with shear rate 0.01-100/s under temperature 25° C. In measurement, a sample is loaded onto the rheometer stage, the probe is lowered, and the sample is allowed to equilibrate for 3 minutes, after which the test is performed. The viscosity at 10/s is reported.

In an aspect, the polyester elastomer (A) used as gel is comprised of particles of size from about 1 μm to about 500 μm as measured by a laser diffraction particle size analyzer. In an aspect, the gel is comprised of particles of size from about 20 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 sm.

In an aspect, the polyester elastomer (A) used as gel is comprised of particles of size with D10 between 10 μm to 50 μm, D50 between 20 μm to 100 μm, and D90 between 30 μm to 200 μm.

The size of particles in polyester elastomer (A) used as gel is measured by HORIBA Scientific Partica LA-960 Laser Scattering Particle Size Analyzer. The sample is prepared by blending 0.3 g of elastomer gel (A) with 15 g of coco-caprylate/caprate and mixing thoroughly at 23° C. A few drops of the diluted gel sample are transferred into the cuvette containing neat coco-caprylate/caprate while constantly stirring. Once the transmittance reaches the acceptable range, the measurement is performed at 23° C. Particle size values of D10, D50 and D90 are reported. The parameter D10 signifies the point in the size distribution, up to and including which, 10% of the total volume of material in the sample is ‘contained’. The parameter D50 signifies the point in the size distribution, up to and including which, 50% of the total volume of material in the sample is ‘contained’. The parameter D90 signifies the point in the size distribution, up to and including which, 90% of the total volume of material in the sample is ‘contained’.

Polyester elastomer (A) as a gel according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980.

The polyester elastomer (A) as gels used according to the invention have an excellent yield point which, for example, has an advantageous effect on their thickening properties and also their ability to stabilize dispersed constituents of personal care formulations. For dynamic oscillation rheology tests, for example a MCR 301 Rheometer (Anton Paar, Graz, Austria) equipped with a 25 mm parallel plate steel geometry can be used.

Polyester elastomer (A) used as gels are notable for the fact that, at a shear rate of 1 l/s and a temperature of 25° C., they have a viscosity of less than 100,000,000 cp and at the same time satisfies G′>G″; Tan-δ<1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the storage modulus (G′) of the polyester elastomer (A) used as gel is from about 10 Pa to about 100,000 Pa as measured by rheometer within linear viscoelastic region using dynamic—42—rheology. In an aspect, the storage modulus (G′) of the gel is from about 100 Pa to about 50,000 Pa. In an aspect, the storage modulus (G′) of the gel is from about 500 Pa to about 30,000 Pa, In an aspect, the storage modulus (G′) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

In an aspect, the loss modulus (G″) of the polyester elastomer (A) used as gel is from about 10 Pa to about 100,000 Pa as measured by rheometer within linear viscoelastic region using dynamic rheology. In an aspect, the loss modulus (G″) of the gel is from about 100 Pa to about 50,000 Pa. In an aspect, the loss modulus (G″) of the gel is from about 500 Pa to about 30,000 Pa, In an aspect, the loss modulus (G″) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

The storage modulus G′ and loss modulus G″ of the polyester elastomer (A) used as gel are measured by Anton Paar rheometer MCR 301, with probe PP25/S at gap 1 mm. The measuring profile is to use flow curve with shear rate 0.01-100/s under temperature 25° C. The measuring profile is to use amplitude sweep with oscillatory strain 0.001-100%, with frequency 1 Hz and under temperature 25° C. In measurement sample is loaded onto the rheometer stage, the probe is lowered and the sample is allowed to equilibrate for 3 minutes after which the amplitude sweep test is performed. The LVR region is determined and the respective G′ value is reported. In an aspect, the polyester elastomer composition is prepared using the methods described herein.

In an aspect, the polyester elastomers described herein are produced using the principles of green chemistry. In an aspect, the polyester elastomers described herein are produced by a simple, efficient environmentally friendly process, with no toxic raw materials used, and no toxic side products generated.

In an aspect, the polyester elastomer gels described herein are produced using the principles of green chemistry.

In an aspect, the polyester elastomer gels described herein are produced by a simple, efficient environmentally friendly process, with no toxic raw materials used, and no toxic side products generated.

Process to Prepare the Composition with Polyester Elastomer Component (A). Solvent or Emollient (B) and Co-Emulsifier (C) as a Gel Composition

In an aspect, the composition is prepared by combining the polyester elastomer component (A), as described herein, with a solvent or emollient (B) as described herein, and with co-emulsifier or emulsion stabilizer (C), as described herein, thereby forming a mixture and shearing the mixture.

In an aspect, the composition is prepared by combining the polyester elastomer component (A), as described herein, with a solvent or emollient (B) as described herein, thereby forming a mixture and shearing the mixture.

In an aspect, the composition is prepared by combining the polyester elastomer component (A), as described herein, and with co-emulsifier or emulsion stabilizer (C), as described herein, thereby forming a mixture and shearing the mixture.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, three roll mill, or a combination thereof.

Subjecting these polyester elastomer compositions to a shearing force produces a polyester elastomer gel which is particular suitable for use in the personal care or cosmetic applications as emulsifier, co-emulsifier and emulsion stabilizer. The claimed cosmetic or personal care applications where this property is most desirable included, but is not limited to, can have for example the form of skin creams, facial creams, hair care products such as conditioners, mousses, and styling gels, protective creams, color cosmetics such as lipsticks, foundations, blushes, makeup, and mascara, and other cosmetic formulations.

In an aspect, the viscosity of the polyester elastomer (A) used in gel form is from about 10 cp to about 1,000,000 cp as measured by rheometer at a shear rate of 0.1 s-1. In an aspect, the viscosity of the gel at 25° C. is from about 30,000 cp to about 900,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, −40-about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

The viscosity of the polyester elastomer gel is measured by Anton Paar rheometer MCR 301, with probe PP25/S at gap 1 mm. The measuring profile is to use flow curve with shear rate 0.01-100/s under temperature 25° C. In measurement, a sample is loaded onto the rheometer stage, the probe is lowered, and the sample is allowed to equilibrate for 3 minutes, after which the test is performed. The viscosity at 10/s is reported.

In an aspect, the polyester elastomer (A) used as gel is comprised of particles of size from about 1 μm to about 500 μm as measured by a laser diffraction particle size analyzer. In an aspect, the gel is comprised of particles of size from about 20 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm.

In an aspect, the polyester elastomer (A) used as gel is comprised of particles of size with D10 between 10 μm to 50 μm, D50 between 20 μm to 100 μm, and D90 between 30 μm to 200 μm.

The size of particles in polyester elastomer (A) used as gel is measured by HORIBA Scientific Partica LA-960 Laser Scattering Particle Size Analyzer. The sample is prepared by blending 0.3 g of elastomer gel (A) with 15 g of coco-caprylate/caprate and mixing thoroughly at 23° C. A few drops of the diluted gel sample are transferred into the cuvette containing neat coco-caprylate/caprate while constantly stirring. Once the transmittance reaches the acceptable range, the measurement is performed at 23° C. Particle size values of D10, D50 and D90 are reported. The parameter D10 signifies the point in the size distribution, up to and including which, 10% of the total volume of material in the sample is ‘contained’. The parameter D50 signifies the point in the size distribution, up to and including which, 50% of the total volume of material in the sample is ‘contained’. The parameter D90 signifies the point in the size distribution, up to and including which, 90% of the total volume of material in the sample is ‘contained’.

Polyester elastomer (A) as a gel according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980.

The polyester elastomer (A) as gels used according to the invention have an excellent yield point which, for example, has an advantageous effect on their thickening properties and also their ability to stabilize dispersed constituents of personal care formulations. For dynamic oscillation rheology tests, for example a MCR 301 Rheometer (Anton Paar, Graz, Austria) equipped with a 25 mm parallel plate steel geometry can be used.

Polyester elastomer (A) used as gels are notable for the fact that, at a shear rate of 1 l/s and a temperature of 25° C., they have a viscosity of less than 100,000,000 cp and at the same time satisfies G′>G″; Tan-δ<1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the storage modulus (G′) of the polyester elastomer (A) used as gel is from about 10 Pa to about 100,000 Pa as measured by rheometer within linear viscoelastic region using dynamic—42—rheology. In an aspect, the storage modulus (G′) of the gel is from about 100 Pa to about 50,000 Pa. In an aspect, the storage modulus (G′) of the gel is from about 500 Pa to about 30,000 Pa, In an aspect, the storage modulus (G′) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

In an aspect, the loss modulus (G″) of the polyester elastomer (A) used as gel is from about 10 Pa to about 100,000 Pa as measured by rheometer within linear viscoelastic region using dynamic rheology. In an aspect, the loss modulus (G″) of the gel is from about 100 Pa to about 50,000 Pa. In an aspect, the loss modulus (G″) of the gel is from about 500 Pa to about 30,000 Pa, In an aspect, the loss modulus (G″) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

The storage modulus G′ and loss modulus G″ of the polyester elastomer (A) used as gel are measured by Anton Paar rheometer MCR 301, with probe PP25/S at gap 1 mm. The measuring profile is to use flow curve with shear rate 0.01-100/s under temperature 25° C. The measuring profile is to use amplitude sweep with oscillatory strain 0.001-100/0, with frequency 1 Hz and under temperature 25° C. In measurement sample is loaded onto the rheometer stage, the probe is lowered and the sample is allowed to equilibrate for 3 minutes after which the amplitude sweep test is performed. The LVR region is determined and the respective G′ value is reported. In an aspect, the polyester elastomer composition is prepared using the methods described herein.

In an aspect, the polyester elastomers described herein are produced using the principles of green chemistry. In an aspect, the polyester elastomers described herein are produced by a simple, efficient environmentally friendly process, with no toxic raw materials used, and no toxic side products generated.

In an aspect, the polyester elastomer gels described herein are produced using the principles of green chemistry.

In an aspect, the polyester elastomer gels described herein are produced by a simple, efficient environmentally friendly process, with no toxic raw materials used, and no toxic side products generated.

### Compositions Used as Emulsifier or Emulsion Stabilizer

Condensation of the starting compounds optionally in presence of one or more solvents or emollients (B) gives the emulsifying polyester elastomer (A) with blocks of hydrophobic backbones with residual carboxylic acid and hydroxyl functional groups, which makes it suitable to be used as an emulsifier, and/or emulsion stabilizer preferably in personal care formulations, specifically in water in oil emulsions.

The polyester emulsifying elastomer component (A) and optionally (B) is suitable for use as emulsifiers and/or emulsion stabilizer particularly in water in oil emulsions e.g. in personal care applications. Personal care emulsion products can take the form of creams, lotions and liquids desirably and typically include emulsifier to aid formation and stability of the emulsion. Typically, emulsion products use the emulsifiers and emulsion stabilizer in amounts of about 0.1 to 50 percent by weight, preferably 1 to about 40% by weight, most preferably 3 to 10% by weight of the emulsion.

The oil phase of such emulsions are typically emollient oils of the type used in personal care or cosmetic products or home care products, which are oily materials which are liquid at ambient temperature or solid at ambient temperature, in bulk usually being a waxy solid, provided they are liquid at an elevated temperature, typically up to 100° C. more usually about 80° C., so such solid emollients desirably have melting temperatures less than 100° C., and usually less than 70° C., at which it can be included in and emulsified in the composition.

The concentration of the oil phase may vary widely and the amount of oil is typically from 1 to 90%, usually 3 to 60%, more usually 5 to 40% by weight of the total emulsion. The amount of water present in the emulsion is typically greater than 5%, usually from 30 to 90%, more usually 50 to 90% by weight of the total composition. 0.1 to 50 percent by weight, preferably 1 to about 40% by weight, most preferably 3 to 10% by weight of the emulsion.

The end use formulations of such emulsions include in the field of personal care products: moisturizers, sunscreens, after sun products, body butters, gel creams, high perfume containing products, perfume creams, baby care products, hair conditioners, hair relaxer formulations, skin toning and skin whitening products, water-free products, antiperspirant and deodorant products, tanning products, cleansers, 2-in-1 foaming emulsions, multiple emulsions, preservative free products, emulsifier free products, mild formulations, scrub formulations e.g. containing solid beads, silicone in water formulations, pigment containing products, sprayable emulsions, colour cosmetics, conditioners, shower products, foaming emulsions, make-up remover, eye make-up remover, and wipes.

Such formulations include green formulations, organic formulations, natural formulations and naturally certified formulations.

In an aspect, the composition with polyester elastomer component (A) is used as emulsifier or emulsion stabilizer.

In an aspect, the composition with polyester elastomer component (A) and solvent or emollient (B) is used as emulsifier or emulsion stabilizer.

In an aspect, the composition with polyester elastomer component (A) and emulsifier or emulsion stabilizer (C) is used as emulsifier or emulsion stabilizer.

In an aspect, the composition with polyester elastomer component (A), solvent or emollient (B), and emulsifier or emulsion stabilizer (C) is used as emulsifier or emulsion stabilizer.

The emulsifying polyester elastomer composition of the invention may also be combined with other emulsifiers and emulsion stabilizers in water-in-oil and oil-in-water emulsions. Alternatively, the ability of emulsifying polyester elastomer composition to function as an emulsifier and/or emulsion stabilizer can be further enhanced by the addition of an interfacially active wax. Preferably, the combination of the novel emulsifying polyester elastomer composition and an interfacially active wax provides an emulsifying wax. Preferably, the emulsifying wax can be substituted for any traditional emulsifying wax in personal care applications.

### Cosmetic or Personal Care Formulations

In an aspect the emulsifier or emulsion stabilizer is for the preparation of emulsions which are cosmetic compositions.

The present invention is also directed to a cosmetic or personal care formulation comprising the emulsifier or emulsion stabilizer.

The present disclosure relates to personal care formulations comprising the emulsifier or emulsion stabilizer for the preparation of emulsions comprising at least one crosslinked polyester component (A), optionally at least one solvent or emollient (B) each as described above, optionally one or more other emulsifier or emulsion stabilizer (C), and other ingredients commonly used in the cosmetic field (D). Components (A), (B), (C) and (D) are each different from each other.

In some aspects, the other ingredients commonly used in the cosmetic field (D) are selected from the group consisting of cleansing agents, hair conditioning agents, skin conditioning agents, hair styling agents, antidandruff agents, hair growth promoters, perfumes, sunscreen compounds, pigments, moisturizers, film formers, humectants, alpha-hydroxy acids, hair colours, make-up agents, detergents, thickening agents, antiseptic agents, deodorant actives and surfactants, cleaning agents such as glass, window, bathroom, kitchen, hard surface cleaning agents, and the like, degreasing agent, vitamins, moisturizers, suspending agents, emulsifiers, preservatives, rheology modifiers, pH adjustor, reducing agents, anti-oxidants, foaming agents, de-foaming agents, chelating agents, gums or thickeners, oils, waxes, fragrances, essential oils, and combinations thereof.

In an aspect, the personal care formulation can be a personal care application, such as a deodorant, an antiperspirant, a skin cream, a facial cream, a hair shampoo, a hair conditioner, a mousse, a hair styling gel, a hair spray, a protective cream, a lipstick, a facial foundations, lushes, makeup, and mascara, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

In an aspect the cosmetic compositions can comprise:
0.01 to 80 percent by weight of the emulsifier or emulsion stabilizer for the preparation of emulsions, comprising:
1.0 to 100% by weight, preferably 20 to 99% by weight of one or more crosslinked polyester components (A),
0 to 99% by weight, preferably 1.0 to 80% by weight of one or more solvent or emollient (B),
based on the total of (A) and (B),
0 to 40%, preferably 1.0 to 40% by weight of one or more other emulsifier or emulsion stabilizer (C),
0 to 60% by weight of one or more other components commonly used in the cosmetic field (D),
each based on the total weight of the composition.

### (Additional) Benefits of Using the Emulsifier or Emulsion Stabilizer as Described Herein, in Personal Care and Cosmetic Formulations.

Due to their outstanding properties the emulsifier or emulsion stabilizer comprising the crosslinked polyester component (A) and optionally one or more solvents or emollients (B) not only provide an emulsifying or emulsion stabilization effect to emulsions but also provide several additional benefits in particular when used as emulsifier or emulsion stabilizer for the manufacture of cosmetic or personal care formulations.

### Surfactant Free Emulsion

Traditional emulsions have been prepared by adding surfactants in order to maintain the dispersion of small droplets in the internal phase during emulsification. Surfactants lower the interfacial tension between the phases and form an interfacial film to prevent irreversible coalescence of the droplets.

There is a negative impact of surfactants on some of the users associated with skin irritation due to a modification of the stratum corneum multilamellar lipid structure. Surfactants can penetrate through the skin barrier and act as lipid solvents. When water is present on the skin, the skin lipids can wash away with the surfactants and reduce the skin barrier defense systems.

An advantage of the present disclosure is the emulsifier or emulsion stabilizer comprising the crosslinked polyester component (A) allows the formation of emulsion that is stable without the use of an additional surfactant, wherein stability of the said emulsion is achieved by hydrogen bonding interactions, ionic repulsion and steric stabilization between in particular a hydroxyl group, a carboxylic acid group and/or an anionic carboxyl groups on the crosslinked polyester elastomer (A).

### Reduce Irritation

A further benefit of the emulsifier or emulsion stabilizer comprising the crosslinked polyester component (A), is to reduce irritation of primary surfactants such as alkyl ether sulphates and alkyl sulphates, for example in baby care formulations or cleaning products for items used by infants. End use formulations of such emulsions include facial cleansers including acne cleansers, shampoos including baby shampoos, 2in1 shampoo with conditioners, facial and body washes, shower gels and shower creams, hand soaps including cream hand soaps.

### Sensory

Water-in-oil emulsions have a significantly better care effect than oil-in-water emulsions. However, it is known to one skilled in the art that water-in-oil emulsions are difficult to stabilize against coalescence of the water droplets and therefore against the separating out of water. To achieve an adequate stability required by the cosmetic's industry, stabilizing waxes, high-viscosity oils, and oil phase thickeners are used. However, stabilizing waxes, high-viscosity oils, and oil phase thickeners have an adverse influence on the sensation on the skin, which manifests itself in a heavy and tacky feeling.

A generally worthwhile aim is thus to make water-in-oil emulsions as light as possible, without having to surrender their particular care effect. Furthermore, there is a need to make water-in-oil emulsions that are stable but not tacky. The emulsifier and emulsion stabilizer play a particular key role in this context.

There is a considerable interest, both among producers and among consumers of emulsion preparations, in W/O emulsifiers and emulsion stabilizers which are based on natural raw materials. Esters of polyalcohols, such as glycerol, polyglycerol, sorbitol or methyl glucoside, and fatty acids, such as oleic or isostearic acid, find diverse uses for this reason. Nevertheless, relatively high concentrations of viscosity-increasing waxes (≥3%) are required for the stabilization, these having an adverse effect on the application properties since they produce an undesirable tacky-greasy feeling on the skins.

The polyglycerol polyricinoleates formed from castor oil by an analogous process are also efficient W/O emulsifiers. However, their sensitivity to oxidation and the often greasy-rancid smell limits their applications in cosmetic or pharmaceutical emulsion preparations.

Herein the emulsifier or emulsion stabilizer comprising the emulsifying polyester elastomer (A) allows formulation of highly liquid emulsions which have a pleasant sensation on the skin. Low-viscosity but stable, water-in-oil and oil-in-water emulsions can be obtained if the emulsifying polyester elastomer composition are used as emulsifiers or emulsion stabilizer.

Oil-based compositions are often oily and tacky in nature and can leave behind an oily residue on skin after application. However, the emulsifier or emulsion stabilizer comprising the crosslinked polyester component (A), in particular, when used as a gel in one or more solvents or emollients as described herein, in cosmetic formulations has additionally significantly improved their sensory properties, adding a feel of silky-smooth skin upon application. Due to its additional thickening effect in formulations, the emulsifier or emulsion stabilizer comprising the crosslinked polyester component (A), in particular, when used as a gel in one or more solvents or emollients (B) as described herein, adds also body and texture to cosmetic composition, providing a cushioning effect. The emulsifier or emulsion stabilizer comprising the crosslinked polyester component (A), in particular, when used as a gel in one or more solvents or emollients (B) as described herein, also improves oil absorption, which minimizes the oily feel of cosmetic compositions. In addition, formulations with the emulsifier or emulsion stabilizer comprising the crosslinked polyester component (A), in particular, when used as a gel in one or more solvents or emollients (B) as described herein, remain on the skin longer than formulations without the crosslinked polyester component (A) forming long-lasting films that are non-tacky.

### Emulsion Stabilization

The water-in-oil emulsions which exhibit good storage stability at low temperatures (i.e. 4° C.), at room temperature (i.e. 25° C.) and at high temperatures (i.e. 45° C.) are high desirable. A significantly improved stabilization of the phase boundaries in emulsions can be achieved by the of the emulsifier or emulsion stabilizer comprising the crosslinked polyester component (A), with both part (A) crosslinked polyester and part (C) other emulsifiers.

The improvement in the resistance to freezing-thawing is of considerable practical interest for the transportability and storability of the emulsion preparations. By relatively long storage at very low temperatures or by extreme variations in temperature over relatively long transportation routes, the inadequate emulsion stabilization can manifest itself by significant separating out of water in the emulsion preparation or can even lead to complete breaking of the emulsion.

It has been found that while strongly polar plant oils are incorporated into formulation, these products with conventional emulsifiers unstable at low temperatures.

Herein the emulsifier or emulsion stabilizer comprising the crosslinked polyester component (A) allows formulation of highly liquid emulsions which improve the emulsion stabilization, especially resistance to freezing-thawing.

### Easy-to-Use

The emulsifier or emulsion stabilizer comprising the crosslinked polyester component (A), when it is a liquid or a gel at room temperature, is also suitable for energy-saving “cold-cold” preparation of emulsions, in addition to the conventional “hot-hot” preparation.

### Spreadability

Possible cosmetic formulations can also acquire an easily spreadable consistency by using the emulsifier or emulsion stabilizer comprising the crosslinked polyester component (A), because by means of these emulsifier systems an oil can be readily incorporated into an aqueous phase or an aqueous phase can be readily incorporated into an oil, for example creams, such as care creams, baby creams or sun protection creams, ointments, lotions or make-up.

### Optical Benefits

In addition to the oily feel of many oil-based formulation, they can often appear shiny or glossy upon application to the skin. However, the emulsifier or emulsion stabilizer comprising the crosslinked polyester component (A), in particular, when used as a gel in one or more solvents or emollients (B) as described herein, also increases oil absorption in cosmetic applications and therefore decreases gloss of the cosmetic substantially. Gloss values of cosmetic compositions formulated with the emulsifier or emulsion stabilizer comprising the crosslinked polyester component (A), in particular, when used as a gel in one or more solvents or emollients (B) as described herein, compared to compositions without it were much lower (measured by the BYK glossmeter). Not only does the crosslinked polyester component (A), in particular, when used as a gel in one or more solvents or emollients (B) as described herein, provide a mattifying effect to the cosmetic formulation, but it also provides a soft-focus effect, blurring lines and improving the optical benefits of the formulation.

Good Compatibility with Cosmetic Ingredients

The emulsifier or emulsion stabilizer comprising the crosslinked polyester component (A) of the present invention has good compatibility with solvent, emollient, film former, pigment, humectant, vitamin, moisturizer, conditioner, oil, suspending agent, surfactant, emulsifier, preservative, rheology modifier, pH adjustor, reducing agent, anti-oxidant, foaming agents, de-foaming agents, chelating agents, gums or thickeners, oils, waxes, a fragrances, essential oils, and combinations thereof.

The emulsifier or emulsion stabilizer comprising the crosslinked polyester component (A) of the present invention has better compatibility than the silicone elastomer gel in ester emollient, natural oil, humectant, vitamin, surfactant, anti-oxidant and combinations thereof.

The emulsifier or emulsion stabilizer comprising the crosslinked polyester component (A) of the present invention has better compatibility than the silicone elastomer gel in UV filters.

The emulsifier or emulsion stabilizer comprising the crosslinked polyester component (A) of the present invention has better compatibility than the silicone elastomer gel in hydrocarbon, mono-estes, di-esters, triglycerides, citrate esters described in more solvents or emollients (B).

### Biodegradability

The emulsifier or emulsion stabilizer comprising the crosslinked polyester component (A), in particular, when used as a gel in one or more solvents or emollients (B) as described herein, is biodegradable. In one aspect, the testing material is above 20% biodegradable within 28 days based on OECD 301B and 301F methods. In another aspect, the testing material is above 20% within 60 days based on OECD 301B and 301F methods. In another aspect, the testing material is above 60% within 60 days based on OECD 301B and 301F methods.

### Skin Nourishment and Conditioning

The emulsifier or emulsion stabilizer comprising the crosslinked polyester component (A) of the present invention are also useful for regulating the condition of the skin and especially for regulating keratinous tissue condition. Regulation of skin condition, namely mammalian and in particular human skin condition, is often required due to conditions which may be induced or caused by factors internal and/or external to the body. Examples include, environmental damage, radiation exposure (including ultraviolet radiation), chronological aging, menopausal status (e.g., post-menopausal changes in skin), stress, diseases, etc. For instance, “regulating skin condition’ includes prophylactically regulating and/or therapeutically regulating skin condition, and may involve one or more of the following benefits: thickening of skin (i.e., building the epidermis and/or dermis and/or sub-dermal (e.g., subcutaneous fat or muscle) layers of the skin and where applicable the keratinous layers of the nail and hair shaft) to reduce skin atrophy, increasing the convolution of the dermal-epidermal border (also known as the rete ridges), preventing loss of skin elasticity (loss, damage and/or inactivation of functional skin elastin) such as elastosis, sagging, loss of skin recoil from deformation; non-melanin skin discoloration such as under eye circles.

Thus in a particular aspect the invention further provides the use of the emulsifier or emulsion stabilizer as described before for the manufacture of a cosmetic or personal care composition.

In particular, there is provided the use of at least one crosslinked polyester component (A), comprising a crosslinked polyester which is reaction product of:
(i) at least one compound selected from a polycarboxylic acid, a polycarboxylic acid ester, and combinations thereof,
(ii) at least one polyol, and
(iii) optionally one or more monofunctional component selected from
(a) a monofunctional carboxylic acid; and
(b) a monofunctional alcohol, and
(B) optionally one or more solvents or emollients,
   as emulsifier or emulsion stabilizer for the preparation of emulsions, especially for the preparation of cosmetic or personal care emulsions, such as those selected from Skin Care Products, such as moisturizers, creams, lotions, and serums; Hair Care Products, such as shampoos, conditioners also for Leave-In Treatments; Sunscreens, such as in the form of Water-in-Oil (W/O) Emulsions and Oil-in-Water (O/W) Emulsions; Cosmetics (Makeup) such as foundations, creams, and makeup removers; anti-aging and treatment products such as anti-aging creams.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Esterification to Prepare Polyester Elastomer Component (A)

In a suitable vessel equipped with agitation, heat, and an ability to distill off water, 200 g dimer acid was added along with 35 g diglycerol and 30 g isosteric acid. Next 30 g squalane was added as solvent. After all ingredients were charged under agitation, the temperature of the mass was raised to 160° C., and water was stripped off as formed. The temperature was held until gelation took place and polymer elastomer was formed. Thereafter the elastomer was broken into a powder by mechanical stirring. The elastomer is above 20% biodegradable within 60 days based on OECD 301B and 301F method.

### Example 2: Preparation of Polyester Elastomer Gel from Polyester Elastomer (A)

After an elastomer powder was produced as in Example 1, which did not separate upon standing, 32.5 g coco-caprylate/caprate was added to 175 g of the elastomer. After the elastomer and solvent mixture sit and swell for 1 hour, it was milled with a 3-roll mill, and then diluted with 300 g coco-caprylate/caprate. The milled elastomer solvent mixture was then homogenized in the reactor vessel to produce a creamy, translucent gel of very smooth consistency, suitable for use in personal care formulations. The elastomer is above 60% biodegradable within 60 days based on OECD 301B and 301F method.

### Example 3: Preparation of Polyester Elastomer Gel with Lecithin from Polyester Elastomer (A)

After an elastomer powder was produced, such as that made in Example 1, which did not separate upon standing, 32.5 g coco-caprylate/caprate was added to 175 g elastomer. After the elastomer and solvent mixture sit and swell for 1 hour, it was milled and then mixed with 300 g coco-caprylate/caprate and 35 g lecithin. The milled elastomer solvent mixture was then homogenized in the reactor vessel to produce a creamy, translucent gel of very smooth consistency, suitable for use in personal care formulations. The elastomer is above 60% biodegradable within 60 days based on OECD 301B and 301F method.

### Example 4: Esterification to Prepare Polyester Elastomer Component (A)

In a suitable vessel equipped with agitation, heat, and an ability to distill off water, 200 g dimer acid was added along with 32 g diglycerol and 28 g oleic acid. After all ingredients were charged under agitation, the temperature of the mass was raised to 160° C., and water was stripped off as formed. The temperature was held until gelation took place and polymer elastomer was formed. Thereafter the elastomer was broken into a powder by mechanical stirring. The elastomer is above 20% biodegradable within 60 days based on OECD 301B and 301F method.

### Example 5: Esterification to Prepare Polyester Elastomer Component (A)

In a suitable vessel equipped with agitation, heat, and an ability to distill off water, 130 g hydrogenated dimer acid was added along with 190 g hydrogenated castor oil. Next 50 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring. The elastomer is above 20% biodegradable within 60 days based on OECD 301B and 301F method.

### Example 6: Esterification to Prepare Polyester Elastomer Component (A)

In a suitable vessel equipped with agitation, heat, and an ability to distill off water, 120 g hydrogenated dimer acid was added along with 80 g hydrogenated castor oil and 60 g dimer diol. Next 130 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring. The elastomer is above 20% biodegradable within 60 days based on OECD 301B and 301F method.

### Example 7: Preparation of Polyester Gel from Polyester Elastomer (A)

After an elastomer powder was produced, such as that made in Example 5, which did not separate upon standing, 50 g coco-caprylate/caprate was added to 170 g elastomer. After the elastomer and solvent mixture sit and swell for 1 hour, it was milled with a 3-roll mill, and then diluted with 600 g coco-caprylate/caprate. The milled elastomer solvent mixture was then homogenized in the reactor vessel to produce a creamy, translucent gel of very smooth consistency, suitable for use in personal care formulations. The elastomer gel is above 20% biodegradable within 60 days based on OECD 301B and 301F method.

### EMULSION EXAMPLES

Instrument used for emulsion examples:
Turbiscan LAB by Formulaction Inc
Sorvall ST8 Centrifuge by ThermoScientific
MQuant pH-indicator strips, 0-14
IKA Eurostar 60 digital overhead mixer equipped with a cowles blade

### Example 8: Emulsions Comprised of Polyester Elastomer Gel and Water

Water in oil (w/o) emulsions were prepared by adding water into the polyester elastomer gel (A) of Example 2 with mixing by an overhead mixer with Cowles blade. Water was added gradually while the Cowles blade was being operated at 500 rpm for a total of 5 minutes upon addition of the entire aqueous phase to form a freshly made emulsion. Table 1 shows the emulsions comprising the polyester elastomer gel (A) of Example 2 with different amounts of water.

The emulsion was considered “Formed” (F) if it looked homogeneous once mixing was stopped and did not immediately phase separate. If the emulsion phase separated immediately once mixing was stopped, it was considered “Not Formed” (NF). The emulsion was considered “Stable” (S) if it passed the centrifugation test described in the following paragraph. If the emulsion was formed, however, it did not pass the centrifugation test, it was considered “Not Stable” (NS).

### Centrifugation Test

4-5 mL of the freshly made emulsions listed in Table 1 were added to a centrifuge tube and loaded into the centrifuge which was set to 4500 rpm for 10 min. Once the centrifugation cycle was completed, the samples were visually examined for any signs of phase separation. The samples passed the test if no visual signs of phase separation were observed and the emulsions tested were considered “stable” (S).

### Centrifugation Test Results

TABLE 1
Formation and stability of w/o emulsions made with
polyester elastomer gel and water

| Trial | 1 | 2 | 3 | 4 | 5 | 6 | 7 | 8 | 9 |
|---|---|---|---|---|---|---|---|---|---|
| Ratio | 90:10 | 80:20 | 70:30 | 60:40 | 50:50 | 40:60 | 30:70 | 20:80 | 10: |
| Elastomer | | | | | | | | | 90 |
| gel (A): | | | | | | | | | |
| water | | | | | | | | | |
| (wt %) | | | | | | | | | |
| Emulsion | F | F | F | F | F | F | F | F | NF |
| F/NF | | | | | | | | | |
| Emulsion | S | S | S | S | S | S | S | S | NS |
| S/NS | | | | | | | | | |

After determining if the emulsions formed and if they were stable by the centrifugation test, the emulsions listed in Table 1 were evaluated by Turbiscan to gain more information on their destabilization mechanism. The protocol for the Turbiscan test is described in the following section.

### Turbiscan Test

20 mL of the freshly made emulsions listed in Table 1 were loaded into a clean turbiscan vial and tapped gently to ensure the sample looked homogeneous and no air bubbles were observed. The vial containing the emulsion was loaded into the Turbiscan instrument and the value for the resulting % backscattering was recorded. The test was repeated over the course of 12 days and the resulting backscattering values were recorded.

### Turbiscan Test Results

The Turbsican test evaluated droplet size (water droplets in oil phase) through measuring % backscattering. A higher backscattering value corresponds to a smaller particle size whereas a lower backscattering value corresponds to a larger particle size. Smaller particle size/larger backscattering value is indicative of more stable emulsion. Decrease in the backscattering value over time is an indication of coalescence taking place in the emulsion sample.

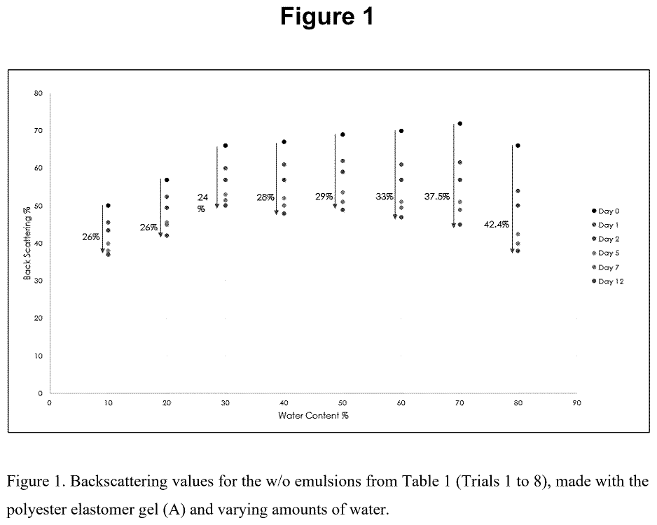

FIG. 1 plots the amount of backscattering (%) on the y-axis as a function of the emulsion's water content on the x-axis as shown in Table 2 indicating also the changes in in BS (backscattering) value from Day 0 to Day 12. The emulsions evaluated are those listed in Table 1 (Trials 1 to 8: Weight ratios Elastomer gel (A): Water (wt %) from 90:10 to 20:80). Each emulsion was analyzed over a period of 12 days; day 0 observed highest % backscattering and the amount of backscattering decreased with each subsequent day. Emulsions containing 50-70 wt % water resulted in the smallest initial particle size, and emulsions containing 10-60 wt % water observed the smallest percent change in the backscattering values over the course of 12 days, hence resulting in the most stable emulsions.

TABLE 2
% Backscattering values for the emulsions made with natural
elastomer gel and DI water (See FIG. 1)

| | | | | | | | | % Change in |
|---|---|---|---|---|---|---|---|---|
| | | BS | BS | BS | BS | BS % | BS % | BS value |
| Trial | Water | % | % | % | % | 7 | 12 | Day 0- |
| # | % | 0 hr | 24 hr | 48 hr | 120 hr | day | day | Day 12 |
| 1 | 10 | 50 | 45.5 | 43.5 | 40 | 38 | 37 | 26 |
| 2 | 20 | 57 | 52.5 | 49.5 | 45.5 | 45 | 42 | 26 |
| 3 | 30 | 66 | 60 | 57 | 53 | 51.5 | 50 | 24 |
| 4 | 40 | 67 | 61 | 57 | 52 | 50 | 48 | 28 |
| 5 | 50 | 69 | 62 | 59 | 53.5 | 51 | 49 | 29 |
| 6 | 60 | 70 | 61 | 57 | 51 | 49.5 | 47 | 33 |
| 7 | 70 | 72 | 61.5 | 57 | 51 | 49 | 45 | 37 |
| 8 | 80 | 66 | 54 | 50 | 42.5 | 40 | 38 | 42 |

### Example 9: Emulsions Comprising of Polyester Elastomer Gel and Water Under Different pH

### Method to Prepare Emulsions Comprising the Polyester Elastomer Gel (A) of Example 2 in Various pH Environments:

The aqueous phase with a desired pH value was prepared by adding a few drops of citric acid or potassium hydroxide and adjusting its pH to the desired value. The pH of aqueous phase was checked with pH strips. The aqueous phase of a certain pH was then added gradually into polyester elastomer gel (A) with the weight ratios indicated in Table 2 with continuing mixing for 4 minutes upon addition of the entire aqueous phase.

As described in Example 8 “F” means emulsion was formed, “S” means the emulsion passed the centrifugation test. “NF” means no emulsion was formed, hence no centrifugation test was performed.

TABLE 2
Effect of pH on the emulsification ability
of the polyester elastomer gel

| | % Aq Phase | | | | | | | |
|---|---|---|---|---|---|---|---|---|
| pH | 10 | 20 | 30 | 40 | 50 | 60 | 70 | 80 |
| 2 | F/S | F/S | F/S | F/S | F/S | F/S | F/S | F/S |
| 4 | F/S | F/S | F/S | F/S | F/S | F/S | F/S | F/S |
| 7 | F/S | F/S | F/S | F/S | F/S | F/S | F/S | F/S |
| 10 | F/S | F/S | F/S | F/S | F/S | NF | NF | NF |
| 12 | F/S | F/S | F/S | F/NS | NF | NF | NF | NF |

Table 2 shows that the aqueous emulsions containing the polyester elastomer gel component (A) are stable over a broad pH and concentration range.

### Example 10: Emulsions Comprising the Polyester Elastomer Gel (A) of Example 2. Water and Emollient (Coco-Caprylate/Caprate)

### Method to Prepare Emulsions Comprising of Polyester Elastomer Gel (A), Water and Emollient (Coco-Caprylate/Caprate):

As described in Example 8 the emulsion was considered “Formed” (F) if it looked homogeneous once mixing was stopped and did not immediately phase separate. The emulsion was considered “Stable” (S) if it passed the centrifugation test. If the emulsion phase separated immediately once mixing was stopped, it was considered “Not Formed” (NF). If the emulsion was formed, however, it did not pass the centrifugation test, it was considered “Not Stable” (NS).

Phase A was prepared by weighing respective amounts of the polyester elastomer gel (A) and coco caprylate/caprate in a beaker. The oil phase was then prepared by mixing it with a cowles blade at 500 rpm for two minutes to ensure homogeneity. Phase B (DI Water) was then added gradually into Phase A with continuing mixing for 3-5 minutes upon addition of the entire aqueous phase B.

TABLE 3
Formation and stability of emulsions made with polyester elastomer
gel, water and emollient (coco-caprylate/caprate)

| Formulation | | 1 | 2 | 3 | 4 | 5 | 6 | 7 | 8 |
|---|---|---|---|---|---|---|---|---|---|
| Phase A | Polyester Elastomer | 22 | 14 | 19 | 13 | 16 | 11 | 14 | 9 |
| | Gel (A), % | | | | | | | | |
| | Coco Caprylate/ | 8 | 6 | 11 | 7 | 14 | 9 | 17 | 11 |
| | Caprate, % | | | | | | | | |
| Phase B | DI Water, % | 70 | 80 | 70 | 80 | 70 | 80 | 70 | 80 |

| | | | | | | | | |
|---|---|---|---|---|---|---|---|---|
| Emulsion Formed/Not Formed | F | F | F | F | F | F | F | F |
| Emulsion Stable/Not Stable | S | S | S | S | NS | S | NS | S |

As shown in Table 3, the polyester elastomer gel (A) can form stable emulsions at only 9% of the total weight.

### Example 11

Aqueous emulsions were prepared according to the following procedure comprising the polyester elastomer gel (A) of Example 2 and Lecithin as component (C) as shown in Table 4, and the stability of the emulsions were at room temperature (23° C.) was observed.

### Procedure:

Mix polyester elastomer (A) with coco-caprylate/caprate and lecithin until homogeneous and homogenize 3 minutes at room temperature (23° C.). Add water into the mixture with mixing by overhead mixer until homogeneous.

TABLE 4

| | Sample | Sample | Sample | Sample | Sample | Sample |
|---|---|---|---|---|---|---|
| Ingredient | A | B | C | D | E | F |
| Water (wt %) | 80 | 80 | 80 | 90 | 90 | 90 |
| Polyester | 7 | 6.8 | 0 | 3 | 2.8 | 0 |
| elastomer (A) | | | | | | |
| (wt %) | | | | | | |
| Coco-caprylate/ | 13 | 13 | 19.8 | 7 | 7 | 9.8 |
| caprate (wt %) | | | | | | |
| Lecithin (wt %) | 0 | 0.2 | 0.2 | 0 | 0.2 | 0.2 |
| Results* | Less | Stable | No | Less | Stable | No |
| | stable | | cream | stable | | cream |
| | than B | | formed | | | formed |

*Tested at 23° C. for 1 week.

As shown in table 4 the polyester elastomer gel (A) of Example 2 shows a synergistic effect with lecithin. Even at high water contents stable emulsions are formed when the polyester elastomer gel (A) is used with lecithin as a further non-ionic emulsifier (C).

### COSMETIC COMPOSITION EXAMPLES

### Example 12—Preparation of Three Water-In-Oil Creams

Three water-in-oil creams are prepared having the compositions shown in Table 5 according to the following procedure:

Mix Phase A and Phase B separately. Slowly add Phase A into Phase B and mix until homogeneous and homogenize 3 minutes. Slowly add mixture of Phase A and Phase B mixture into Phase C and mix until homogeneous.

TABLE 5

| | | | Wt | Wt | Wt |
|---|---|---|---|---|---|
| | | | (%) | (%) | (%) |
| Phase | Ingredients | INCI Name | A | B | C |
| A | Water | Water | To:100 | To:100 | To:100 |
| | NaCl | NaCl | 0.50 | 0.50 | 0.50 |
| | Glycerin | Glycerin | 15.00 | 15.00 | 15.00 |
| B | Sepinov | Hydroxyethyl | 0.05 | 0.05 | 0.05 |
| | EMT10 | acrylate & | | | |
| | | Sodium | | | |
| | | Acryloyldimethyl | | | |
| | | Taurate | | | |
| | | copolymer | | | |
| | Emulsifying Polyester Elastomer (A) (Example 2) | | 8.00 | 8.00 | 8.00 |
| | Liponate^{†} | Coco- | 8.00 | 8.00 | 8.00 |
| | CCC | Caprylate/Caprate | | | |
| | HARMONIE | C9-12 Alkane | 5.00 | 5.00 | 5.00 |
| | Soft fluid | | | | |
| | CITHROL | Polyglyceryl-3 | / | 0.50 | / |
| | PG321S | Diisostearate | | | |
| | CITHROL | Polyglyceryl-3 | / | / | 0.50 |
| | PG3PR | Polyricinoleate | | | |
| C | Preservative | Preservative | q.s. | q.s. | q.s. |
| | Fragrance | Fragrance | q.s. | q.s. | q.s. |

### Example 13

Five oil-in-water creams are prepared having the compositions shown in Table 6 according to the following procedure:

Combine Phase A ingredients and mix in a vessel until uniform at 70° C. Combine Phase B & C and slowly add to Phase A and mix under regular stirring until homogeneous. Cool and add Phase D to Phase ABC under mixing and mix until homogeneous.

TABLE 6

| | | | Wt % | Wt % | Wt % | Wt % | Wt % |
|---|---|---|---|---|---|---|---|
| Phase | Ingredients | INCI Name | A | B | C | D | E |
| A | Emulsifying Polyester Elastomer (Example 2) | | 8.00 | 8.00 | 8.00 | 8.00 | 8.00 |
| | MONTANOVE 68 | Cetearyl Alcohol & | 1.50 | / | / | / | / |
| | | Cetearyl Glucoside | | | | | |
| | MONTANOVE 82 | Cetearyl Alcohol & | / | 1.50 | / | / | / |
| | | Coco-Glucoside | | | | | |
| | MONTANOVE S | Coco-Glucoside & | / | / | 1.50 | / | / |
| | | Coconut Alcohol | | | | | |
| | EMULIUM | Polyglyceryl-6 | / | / | / | 1.50 | / |
| | DERMOLEA MB | Distearate(and)Candelilla/ | | | | | |
| | | Jojoba/Rice bran | | | | | |
| | | Polyglyceryl | | | | | |
| | BMX5000TC | Lecithin | / | / | / | / | 1.50 |
| | CETEARYL | Cetearyl Alcohol | 1.00 | 1.00 | 1.00 | 1.00 | 1.00 |
| | ALCOHOL | | | | | | |
| | NEOSSANCE | Squalane | 5.00 | 5.00 | 5.00 | 5.00 | 5.00 |
| | SIMULSOL 165 | Glyceryl | 1.00 | 1.00 | 1.00 | 1.00 | 1.00 |
| | | Stearate(and)PEG-100 | | | | | |
| | | Stearate | | | | | |
| | Liponate CCC^{(c)} | Coco-Caprylate/Caprate | 8.00 | 8.00 | 8.00 | 8.00 | 8.00 |
| B | Glycerin | Glycerin | 5.00 | 5.00 | 5.00 | 5.00 | 5.00 |
| | Water | Water | To: | To: | To: | To: | To: |
| | | | 100 | 100 | 100 | 100 | 100 |
| C | SEPIPLUS 400 | Polyacrylate-13 and | 0.90 | 0.90 | 0.90 | 0.90 | 0.90 |
| | | Polyisobutene and | | | | | |
| | | Polysorbate 20 | | | | | |
| D | Fragrance | Fragrance | q.s. | q.s. | q.s. | q.s. | q.s. |
| | Preservative | Preservative | q.s. | q.s. | q.s. | q.s. | q.s. |

### Example 14

Water-in-oil lotion creams are prepared having the compositions shown in Table 5 according to the following procedure:

Mix Phase A and Phase B separately. Slowly add Phase A into Phase B and mix until homogeneous.

TABLE 7

| Phase | Ingredients | INCI Name | Wt % |
|---|---|---|---|
| A | Water | Water | 47.0 |
| | Sodium Chloride | Sodium Chloride | 1.0 |
| | Glycerin | Glycerin | 2.0 |
| B | MCT oil | Caprylic/Capric Triglyceride | 40.0 |
| | Emulsifying Polyester Elastomer (Example 2) | | 5.0 |
| | Bentone Luxe WO | Polyglyceryl-6 Polyricinoleate (and) | 5.0 |
| | | Polyglyceryl-3 Diisostearate (and) | |
| | | Disteardimonium Hectorite | |

1. An emulsifier or an emulsion stabilizer comprising:
at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol.

2. The emulsifier or emulsion stabilizer of claim 1, comprising:
at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof,
(ii) at least one polyol, and
(iii) one or more monofunctional component selected from
(a) a monofunctional carboxylic acid; and
(b) a monofunctional alcohol, and
one or more emulsifiers or emulsion stabilizers, different from the at least one crosslinked polyester component.

3. The emulsifier or emulsion stabilizer of claim 1, wherein
the at least one compound (i) is selected from the group consisting of
(1) one or more poly-carboxylic acids of formula (I) 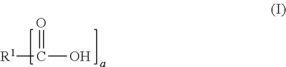
wherein
R¹ is selected from the group consisting of C2-C52 alkyl group, C2-C52 heteroalkyl group, C2-C52 alkene group, C2-C52 heteroalkene group, C3-C52 cyclic group, and C2-C52 heterocyclic group, and
a is an integer from 2 to 10,
(2) one or more poly-carboxylic acid esters of formula (II) 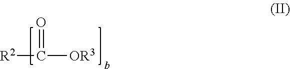
wherein
R² is C1-C22 alkyl group, C2-C22 alkylene group, or C3-C22 cyclic group;
R³ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
b is an integer from 2 to 10, and
(3) a combination thereof,
the at least one polyol (ii) is a polyol of formula (III) 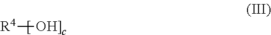
wherein
R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
c is an integer from 2 to 10.

4. The emulsifier or emulsion stabilizer of claim 1, wherein:
the at least one compound (i) is one or more poly-carboxylic acids selected from the group consisting of di-carboxylic acids, tri-carboxylic acids, and combinations thereof;
the at least one polyol (ii) is selected from the group consisting of diols, triols, and combinations thereof.

5.-6. (canceled)

7. The emulsifier or emulsion stabilizer of claim 1, wherein the at least one crosslinked polyester component has a gel fraction which is greater than 20%.

8. The emulsifier or emulsion stabilizer of claim 1, wherein the at least one crosslinked polyester component has a swelling ratio of from about 1 gram/gram to about 15 gram/gram.

9. The emulsifier or emulsion stabilizer of claim 1,
wherein the at least one crosslinked polyester component is comprised of particles of a size from about 1 μm to about 500 μm as measured by a laser diffraction particle size analyzer.

10. The emulsifier or emulsion stabilizer of claim 1, further comprising one or more solvents or emollients, wherein the one or more solvents or emollients are selected from the group consisting of a triglyceride solvent, a mono-ester solvent, a di-ester solvent, a citrate ester solvent, an ether solvent, a carbonate solvent, a hydrocarbon solvent, a silicone solvent, and combinations thereof.

11. The emulsifier or emulsion stabilizer of claim 10, wherein the one or more solvents or emollients is selected from the group consisting of
a triglyceride of formula (XIV) 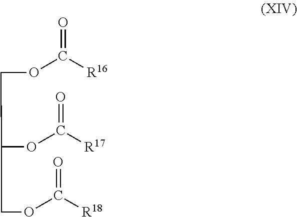
wherein
each R¹⁶, R¹⁷, and R¹⁸ are independently C1-C35 alkyl group, C1-C35 heteroalkyl group, C2-C35 alkene group, or C2-C35 heteroalkene group;
a mono-ester of formula (XV) 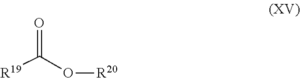
wherein
R¹⁹ is C1-C35 alkyl group, C1-C35 heteroalkyl group, C2-C35 alkene group, or C2-C35 heteroalkene group; and
R²⁰ is C1-C35 alkyl group, C1-C35 heteroalkyl group, C2-C35 alkene group, or C2-C35 heteroalkene group;
a di-ester of formula (XVI) 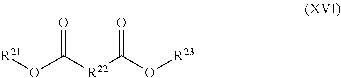
wherein
R²¹ is C1-C35 alkyl group, C1-C35 heteroalkyl group, C2-C35 alkene group, or C2-C35 heteroalkene group; and
R²² and R²³ are independently C1-C35 alkyl group, C1-C35 heteroalkyl group, C2-C35 alkene group, or C2-C35 heteroalkene group;
a di-ester of formula (XVII) 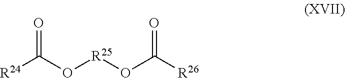
wherein
R²⁴ is C1-C35 alkyl group, C1-C35 heteroalkyl group, C2-C35 alkene group, or C2-C35 heteroalkene group; and
R²⁵ and R²⁶ are independently H, C1-C35 alkyl group, C1-C35 heteroalkyl group, C2-C35 alkene group, or C2-C35 heteroalkene group;
a di-ester of formula (XVIII) 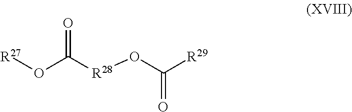
wherein
R²⁷ and R²⁸ are independently C1-C35 alkyl group, C1-C35 heteroalkyl group, C2-C35 alkene group, or C2-C35 heteroalkene group; and
R²⁹ is H, C1-C35 alkyl group, C1-C35 heteroalkyl group, C2-C35 alkene group, or C2-C35 heteroalkene group;
a citrate ester solvent of formula (XIX) 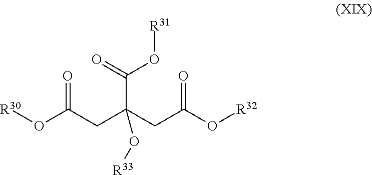
wherein
R³⁰, R³¹, R³², and R³³ are independently H, C1-C35 alkyl group, C1-C35 heteroalkyl group, C2-C35 alkene group, or C2-C35 heteroalkene group, wherein at least one of R³⁰, R³¹, R³², and R³³ is not H;
an ether solvent of formula (XX) 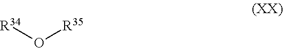
wherein
R³⁴ and R³⁵ are independently H, C2-C20 alkyl group, C2-C20 heteroalkyl group, C2-C20 alkene group, or C2-C20 heteroalkene group, wherein at least one of R³⁴ and R³⁵ is not H;
a carbonate solvent of formula (XXI) 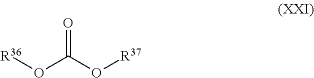
wherein
R³⁶ and R³⁷ are independently H, C2-C20 alkyl group, C2-C20 heteroalkyl group, C2-C20 alkene group, or C2-C20 heteroalkene group;
a hydrocarbon solvent with number of carbon atoms from C4 to C60; and
a silicone solvent.

12. The emulsifier or emulsion stabilizer of claim 2, wherein the one or more emulsifiers or emulsion stabilizers, different from the at least one crosslinked polyester component are selected from the group consisting of
Non-Ionic Emulsifiers and Surfactants;
(iv) Anionic Emulsifiers and Surfactants;
Cationic Emulsifiers and Surfactants
(vi);
(vii) Fatty Acid Derivatives;
(viii) Polyethylene Glycol (PEG) Esters;
and combinations thereof.

13. The emulsifier or emulsion stabilizer of claim 2, wherein the one or more emulsifiers or emulsion stabilizers, different from the at least one crosslinked polyester component are selected from the group consisting of glyceryl esters, diglyceryl esters, polyglyceryl esters, sorbitan or sorbitol ester, sugar ester, lecithin, glycolipids, amino acid derivatives, alkyl polyglycosides, polyester made by reacting diglycerol or polyglycerol having from 3 to 20 glycerol units and polyhydroxystearate or polyricinoleate, polyester made by reacting diglycerol or polyglycerol having from 3 to 20 glycerol units and succinic acid, sebacic acid or dimer acid (dilinoleic acid), and combinations thereof.

14. The emulsifier or emulsion stabilizer of claim 1, wherein the at least one crosslinked polyester component is obtained by a process comprising:
(a) reacting the at least one compound (i) and the at least one polyol (ii)
to obtain the crosslinked polyester component,
(b) subjecting the crosslinked polyester obtained in (a) to shear forge to obtain the crosslinked polyester component, or
(c) adding one or more solvents to the crosslinked polyester component obtained in (a), allowing the resulting mixture to swell in the one or more solvents, subjecting said swollen mixture to shear force to obtain the at least one crosslinked polyester component as a gel in the one or more solvents.

15. The emulsifier or emulsion stabilizer of claim 14, wherein the shear force in (b) or (c) is carried out with a batch mixer, planetary mixer, single or multiple screw extruder, dynamic or static mixer, colloid mill, homogenizer, sonolator, three roll mill, or a combination thereof.

16. The emulsifier or emulsion stabilizer of claim 14, wherein the one or more solvents in (c) is such that the resulting crosslinked polyester component comprises from about 10% to about 95% weight by weight of the one or more solvents.

17. (canceled)

18. A personal care composition comprising one or more of the emulsifier or emulsion stabilizer of claim 2, and
one or more components commonly used in the cosmetic field, different from the at least one crosslinked polyester component.

19. The personal care composition of claim 18, wherein the one or more components used in the cosmetic field is selected from the group consisting of water, solvent, emollient, film former, pigment, humectant, vitamin, moisturizer, conditioner, oil, suspending agent, surfactant, emulsifier, preservative, rheology modifier, pH adjustor, reducing agent, anti-oxidant, foaming agents, de-foaming agents, chelating agents, gums or thickeners, oils, waxes, a fragrances, essential oils, and combinations thereof.

20. The personal care composition of claim 18, which is an aqueous emulsion, selected from a water-in-oil emulsion and an oil-in-water emulsion.

21. (canceled)

22. The personal care composition of claim 18, comprising about 0.01 to 50% by weight of the at least one crosslinked polyester component based on the total weight of the composition.

23. (canceled)

24. The personal care composition of claim 18, which is selected from the group consisting of a deodorant, an antiperspirant, a skin cream, a facial cream, a hair shampoo, a hair conditioner, a mousse, a hair styling gel, a hair spray, a protective cream, a lipstick, a facial foundation, a blush, makeup, a mascara, a skin care lotion, a moisturizer, a facial treatment, a personal cleanser, a facial cleanser, a shaving cream, a pre-shave lotion, an after-shave lotion, and a sunscreen.

25.-28. (canceled)

29. The emulsifier or emulsion stabilizer of claim 1, which is at least 20% biodegradable within 60 days based on OECD 301B and 301F test.

30. The emulsifier or emulsion stabilizer of claim 2, wherein:
the monofunctional carboxylic acid (a) is a mono-carboxylic acid of formula (IV) 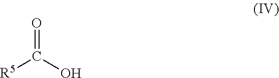
wherein
R⁵ is C2-C52 alkyl group, C2-C52 heteroalkyl group, C2-C52 alkene group, C2-C52 heteroalkene group, C3-C52 cyclic group, or C2-C52 heterocyclic group; and
the monofunctional alcohol of (b) is a mono-alcohol of formula (V) 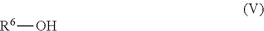
wherein
R⁶ is C2-C52 alkyl group, C2-C52 heteroalkyl group, C2-C52 alkene group, C2-C52 heteroalkene group, C3-C52 cyclic group, or C2-C52 heterocyclic group.
